(19) United States
(12) Patent Application Publication
(10) Pub. No.: US 2013/0116215 A1
(43) Pub. Date: May 9, 2013

(54) COMBINATION THERAPIES FOR TREATING NEUROLOGICAL DISORDERS

(71) Applicants: Mireia Coma, Vic (ES); Patrick Aloy, Alella (ES); Albert Pujol, El Masnou (ES); Xavier Gomis, Sant-Just Desvern (ES); Baldomero Oliva, Terrassa (ES); Alberto Lleó, Barcelona (ES); José Manuel Mas, Barcelona (ES)

(72) Inventors: Mireia Coma, Vic (ES); Patrick Aloy, Alella (ES); Albert Pujol, El Masnou (ES); Xavier Gomis, Sant-Just Desvern (ES); Baldomero Oliva, Terrassa (ES); Alberto Lleó, Barcelona (ES); José Manuel Mas, Barcelona (ES)

(21) Appl. No.: 13/660,205

(22) Filed: Oct. 25, 2012

Related U.S. Application Data
(60) Provisional application No. 61/552,922, filed on Oct. 28, 2011.

(51) Int. Cl. A61K 45/06; A61K 31/40; A61K 31/549; A61K 31/51; A61K 31/48; A61K 31/663; A61K 31/4422; A61K 31/4174; A61K 31/4045; A61K 31/337; A61K 31/196; A61K 31/5377; A61K 31/655; A61K 31/4406; A61K 31/165; A61K 31/56; A61K 31/428

(52) U.S. Cl. CPC A61K 45/06; A61K 31/428; A61K 31/40; A61K 31/549; A61K 31/51; A61K 31/48; A61K 31/663; A61K 31/4422; A61K 31/4174; A61K 31/4045; A61K 31/337; A61K 31/196; A61K 31/5377; A61K 31/655; A61K 31/4406; A61K 31/165; A61K 31/56
USPC 514/108; 514/367; 514/428; 514/223.5; 514/276; 514/288; 514/236.5; 514/151; 514/357; 514/171

(57) ABSTRACT

The invention features novel pharmaceutical combinations useful for the treatment of neurological diseases, specifically neurodegenerative diseases. The novel pharmaceutical combinations of the invention demonstrate additive or synergistic effect in silico and in vivo. The invention also relates to methods of treatment of neurological and neurodegenerative diseases including the pharmaceutical combinations of the invention.

This application claims the benefit of U.S. Provisional Application No. 61/552,922, filed Oct. 28, 2011.

### BACKGROUND OF THE INVENTION

Neurodegenerative Diseases

Nervous System Diseases, specially neurodegenerative diseases such as Parkinson Disease, Tauopathies, Alzheimer's Disease (AD), Diffuse Neurofibrillary Tangles with Calcification, Supranuclear Palsy, Progressive, TDP-43 Proteinopathies, Amyotrophic Lateral Sclerosis, Frontotemporal Lobar Degeneration, Lewy Body Disease, AIDS Dementia Complex, Aphasia, Primary Progressive, Primary Progressive Nonfluent Aphasia, Dementia, Vascular, CADASIL, Dementia, Multi-Infarct, Diffuse Neurofibrillary Tangles with Calcification, Frontotemporal Lobar Degeneration, Frontotemporal Dementia, Primary Progressive Nonfluent Aphasia, Kluver-Bucy Syndrome, Pick's Disease, Motor Neuron Disease, Amyotrophic Lateral Sclerosis, Bulbar Palsy, Progressive, Muscular Atrophy, Spinal, Multiple System Atrophy, Olivopontocerebellar Atrophies, Shy-Drager Syndrome, Striatonigral Degeneration, Olivopontocerebellar Atrophies, Paraneoplastic Syndromes, Nervous System, Lambert-Eaton Myasthenic Syndrome, Limbic Encephalitis, Myelitis, Transverse, Opsoclonus-Myoclonus Syndrome, Paraneoplastic Cerebellar Degeneration, Paraneoplastic Polyneuropathy, Postpoliomyelitis Syndrome, Prion Diseases, Encephalopathy, Bovine Spongiform, Gerstmann-Straussler-Scheinker Disease, Insomnia, Fatal Familial, Kuru, Scrapie, Wasting Disease, Chronic, Creutzfeldt-Jakob Syndrome, Shy-Drager Syndrome, Subacute Combined Degeneration, Heredodegenerative Disorders, Nervous System, Alexander Disease, Amyloid Neuropathies, Familial, Bulbo-Spinal Atrophy, X-Linked, Canavan Disease, Cockayne Syndrome, Dystonia Musculorum Deformans, Gerstmann-Straussler-Scheinker Disease, Hepatolenticular Degeneration, Hereditary Central Nervous System Demyelinating Diseases, Hereditary Sensory and Autonomic Neuropathies, Hereditary Sensory and Motor Neuropathy, Huntington Disease, Lafora Disease, Lesch-Nyhan Syndrome, Menkes Kinky Hair Syndrome, Myotonia Congenita, Myotonic Dystrophy, Neurofibromatoses, Neuronal Ceroid-Lipofuscinoses, Optic Atrophies, Hereditary, Pantothenate Kinase-Associated Neurodegeneration, Rett Syndrome, Spinal Muscular Atrophies of Childhood, Spinocerebellar Degenerations, Tourette Syndrome, Tuberous Sclerosis, Unverricht-Lundborg Syndrome and others, are major causes of disease in the world [1].

There is a worldwide rising trend of neurodegenerative diseases. Alzheimer's disease, the most common neurodegenerative disease, has the exponential increase of its prevalence between 65 and 85, doubling every 5-year of age in developed countries. It is currently estimated to affect 35 million worldlife in 2010, with an expected increase to 113 million by the year 2050 [2] [3]. The increasing life expectancy in the last years has led to an increase in the prevalence of this age-related condition and has posed an important medical and social challenge for developed societies.

Neurodegenerative disorders are hereditary and/or idiopathic conditions characterized by progressive nervous system dysfunction that result in progressive degeneration and/or death of nerve cells. Their etiology is not yet fully understood. However, evidence for a complex interplay between several mechanisms as energy metabolism, excitotoxicity, protein aggregation, oxidative damage is increasingly compelling, which highlights the potential value of complex molecular interaction profiles in the discovery of novel multicomponent therapies.

Research into Neurodegenerative disease therapy has been at least partly successful in terms of developing symptomatic treatments, but has also had several failures in terms of developing disease-modifying therapies as per example in Amyotrophic Lateral Sclerosis, Alzheimer's disease and Pick's disease. The only FDA approved drugs for the treatment of Alzheimer's disease are inhibitors of AChE (tacrine (Cognex), donepezil (Aricept), rivastigmine (Exelon), and galantamine (Reminyl)); and the N-methyl D-aspartate (NMDA) antagonist memantine (Namenda). These drugs produce modest symptomatic benefit on cognitive, behavioral and functional symptoms with minimal impact on the disease process [4].

New treatments are needed, and current research is failing to provide and help understand mechanisms of action and to suggest new targets or compounds. Recently, a large number of clinical trials have failed as per example AZD3480, an orally active neuronal nicotinic agonist or Eptastigmine a long-acting cholinesterase inhibitor both due to lack of efficacy or AN1792, an active immunotherapy with Aβ1-42 peptide due to lack of safety.

Successes and failures have led to debate about the potential deficiencies in our understanding of the pathogenesis of neurodegenerative diseases and potential pitfalls in development of drug candidates and the correct choice of therapeutic targets.

Neurodegenerative diseases are complex in origin, with multiple molecular interactions involving both host genomes and environmental determinants, with metabolic interactions between multiple cell types as neurons, microglia, astroglia, vascular system and others. It is more and more clear that multi-target polypharmacological research is needed to interact with different targets and modify different molecular pathways. The discovery of drug combinations and the understanding of their complex modes of action will outline an avenue of therapies against neurodegenerative diseases [5] [6] [7]. Clinical success with multicomponents therapies and multi-targeted agents have been shown in other pathologies like asthma [8], hyperlipidemia [9], HIV-1 [10] or cancer [11] [12].

Knowledge of the molecular mechanisms of drug and multicomponent therapies are also being investigated in neurodegenerative diseases and others complex diseases [3, 7]. For example, Alzheimer's disease is complex in nature, with several mechanism involved as amyloid formation, aggregation, degradation or clearance, tau phosphorilation and aggregation, oxidative stress, excitotoxicity, energy metabolism and inflammation in which not only neurons also microglia, astroglia, vascular smooth muscle cells and endothelial cells are implicated.

Use of New Systems Biology Technologies and Methods for Drug Discovery

Recently, the company Anaxomics has developed a new strategy to discover new useful compounds by new technologies in Systems Biology, described in Patent application WO 2011/051805 and elsewhere [5] [13] [14]. Specifically Anaxomics has discovered new drug discovery methods, and specially methods to discover new drug-drug combinations or polypharmacological approaches with surprising and unexpected therapeutic effects.

These methods are based in a new methodology, The Therapeutic Performance Mapping System (TPMS) technology, which is used for the prediction of safety and efficacy characteristics of medicines, by using the full potential of Systems Biology approaches to understand and model the complex mechanism of action of drugs.

The goal of Systems Biology technologies is to understand the full complexity of biological systems by using all possible available data coming from “omic” sources (genomics, transcriptomics, proteomics, metabolomics, interactomics, pharmacogenomics and others), combined with the known data about clinical effects of drugs (therapeutic effects and adverse events). Novel mathematical models and predictive strategies are then developed which can be used to make predictions of the future behavior of drugs or compounds in a given organism.

This novel method for drug discovery includes a 3 step process; (1) Map creation, (2) Developing mathematical methods, (3) Data analysis and experimental checking (FIG. 1).

The first step is to focus the global Map of molecular interactions, typically protein-to-protein interactions organized in metabolic pathways, around drug targets or around the key proteins of a pathological condition that define the system of analysis. This step includes the use of a Biological Effectors Database, that links observable clinical phenotypes (i.e., adverse events and therapeutic indications), with its physiological mechanisms and with the individual proteins or subnetworks of proteins that are related with these mechanisms, and in consequence, with the phenotypic observations of interest.

The second step consists on the creation, validation, refine and check of mathematical models by using known data about targets, mechanisms of action of drugs, and their clinical observable effects, usually those biological effects, to emulate human disease physiology. The mathematical model of the map will be developed by means of rules, any type of artificial intelligence learning process, supervised or not, genetic algorithms, artificial neural networks of any type and variant or stochastic methods like Simulated Annealing, Montecarlo or whatever similar method known.

The third step, Data Analysis and Experimental Checking, allows the generation of mechanistic hypothesis related to avoiding or predicting drug AEs, propose new indications and improve on the understanding of drug mechanism of action. The experimental validation of the results predicted are conducted by using biological data, by “In vitro” and “In vivo” studies, by “in silico” simulations, as well as by expert Literature Search on indexed PubMed articles.

Reliability of the Predictive Methods

The TPMS technology used for the discovery of the drug-drug combinations disclosed herein has been proven to provide reliable predictions in terms of future positive results in disease models of drug-drug combinations, in a number of diseases and situations. For instance, one example of the reliability of the predicted methods used for the discovery of the drug-drug combinations disclosed herein, is the discovery of the neurological effects of proton pump inhibitors (PPIs) used typically in the treatment of peptic ulcer disease and in other conditions where inhibition of gastric acid secretion may be beneficial. Interestingly, this technology has detected an unexpected association of PPIs, and in particular Lansoprazole, with Alzheimer's disease and with R-amyloid pathology, by using one type of mathematical analysis and models covered in the TPMS technology. This neurological effect of the Lansoprazole was not described in any prior art for this drug and neither for its targets. This fact is a relevant new relationship that could be especially important for patients suffering from Alzheimer's disease.

The putative neurological effects of this drug have been confirmed experimentally using sensitive Aβ1-40 and Aβ1-42 ELISA kits (Wako, Osaka, Japan) on the extracellular media of treated and untreated cells stably expressing wild-type presenilin-1 an Aβ precursor protein. Lansoprazole significantly increases Aβ peptide production, both Aβ1-40 and Aβ1-42. Aggregation and fibril formation of Aβ peptides are central events in the pathogenesis of Alzheimer's disease [15]. Previous studies have established the ratio of Aβ1-40 to Aβ1-42 as an important factor in determining the fibrillogenesis, and toxicity of amyloid plaques. Interestingly, a significant reduction of Aβ1-40/Aβ1-42 ratio has been observed on Lansoprazole treated cells which further confirm the potential negative effect of Lansoprazole on memory (FIG. 2).

Thus in silico predictive efficacy of TPMS technology is accepted in the drug discovery field as having a strong correlation with future therapeutic efficacy in disease in vitro models commonly accepted in the field. The TPMS drug discovery methodology is the methodology applied herein for the discovery of the compounds and combinations described, and the predictive positive efficacy and/or safety of the therapeutic combinations discovered by using such methods is one of the key evidences of the efficacy and/or safety of the therapeutic combinations. The experimental disease model evidences obtained for combinations described herein provide confirmation of the positive predictive value for the TPMS discovery methodology.

### SUMMARY OF THE INVENTION

The invention discloses novel drug-drug combinations, pharmaceutical compositions, kits and treatment methods for the treatment or prevention of neurodegenerative diseases.

By using new systems biology computing technologies described elsewhere, the inventors have discovered certain drug-drug combinations with unexpected and surprising useful benefits for the treatment of neurological and neurodegenerative diseases. The utility of the new drug-drug combinations is unexpected and surprising because the combinations described herein have not been described previously as useful for the treatment of neurological or neurodegenerative diseases.

This invention provides new pharmacological combinations and pharmaceutical compositions comprising at least two or more compounds or an acceptable salt thereof, selected from the group including L-Glutamine, Biotin, L-Lysine, Vitamin C, L-Leucine, L-Methionine, L-Alanine, L-Isoleucine, Methadone, Methoxyflurane, Tacrolimus, Alfentanil, Aspirin, Halothane, Danazol, Estriol, Acetic Acid, Adenosine monophosphate, Arsenic trioxide, Atropine, Azelaic Acid, Chloroprocaine, Dimethyl sulfoxide, Ethanol, Fludarabine, Fomepizole, Isoflurane, L-Carnitine, Praziquantel, Promethazine, Rifampin, Spermine, Terfenadine, Vitamin E, Acarbose, Acetohydroxamic Acid, Aciclovir, Adenine, Adenosine triphosphate, Alclometasone, Alemtuzumab, Alendronate, Alpha-Linolenic Acid, Amifostine, Amlexanox, Amlodipine, Amodiaquine, Amrinone, Aspartame, Astemizole, Atazanavir, Atorvastatin, Atovaquone, Bacitracin, Balsalazide, Beclomethasone, Buclizine, Calcitriol, Cefadroxil, Cefalotin, Cefazolin, Cefdinir, Cefepime, Cefonicid, Cefoperazone, Cetuximab, Chloramphenicol, Chlorpheniramine, Cinnarizine, Ciprofloxacin, Clofarabine, Clopidogrel, Clotrimazole, Cloxacillin, Cocaine, Cyanocobalamin, Cyclizine, Cycloserine, Cyclosporine, Cyclothiazide, Cyproheptadine, Dapsone, Daunorubicin, Diethylstilbestrol, Dipyridamole, Disulfuram, Dofetilide, Enflurane, Enfuvirtide, Enoxacin, Enprofylline, Epinastine, Ertapenem, Ethchlorvynol, Ezetimibe, Felodipine, Fenofibrate, Flucytosine, Flumethasone Pivalate, Flunisolide, Fluorouracil, Fluticasone Propionate, Folic Acid, Framycetin, gamma-Homolinolenic acid, Gatifloxacin, Gemcitabine, Gentamicin, Glibenclamide, Glucosamine, Glutathione, Gonadorelin, Guanidine, Halobetasol Propionate, Halofantrine, Heparin, Hesperetin, Histamine Phosphate, Human Serum Albumin, Hyaluronidase, Hydroxocobalamin, Ibutilide, Icosapent, Iloprost, Imatinib, Irinotecan, Isoniazid, Isradipine, Itraconazole, L-Arginine, L-Aspartic Acid, L-Citrulline, L-Cysteine, L-Cystine, Levamisole, Levocabastine, Levofloxacin, Levothyroxine, Lidocaine, Lomefloxacin, L-Ornithine, L-Proline, L-Threonine, Menadione, Miconazole, Minocycline, Mitotane, Montelukast, Moxifloxacin, Mycophenolic acid, Nafarelin, Nedocromil, Niacin, Nitrendipine, Nitrofurazone, Nitroglycerin, Norfloxacin, Ofloxacin, Olopatadine, Oseltamivir, Palivizumab, Pefloxacin, Pegademase bovine, Penicillamine, Phentolamine, Potassium Chloride, Pranlukast, Pravastatin, Probenecid, Procaine, Proguanil, Pyrazinamide, Pyrimethamine, Pyruvic acid, Quinacrine, Quinine, Ribavirin, Riboflavin, Rifabutin, Rimexolone, Roxithromycin, Saquinavir, Sevoflurane, Sparfloxacin, Succinic acid, Sulfinpyrazone, Sulfisoxazole, Sulindac, Tetracycline, Tetrahydrofolic acid, Theophylline, Topotecan, Tretinoin, Triamcinolone, Trifluridine, Trimethoprim, Trovafloxacin, Urokinase, Verapamil, Vidarabine, Vitamin A, Vorinostat, Warfarin, Xanthophyll, Zanamivir, L-Histidine, Choline, Glycine, L-Tryptophan, L-Serine, NADH, Tramadol, Caffeine, Lisdexamfetamine, Methamphetamine, Methoxamine, Prazosin, Lorazepam, Butorphanol, Codeine, Hydrocodone, Hydromorphone, Morphine, Nalbuphine, Oxycodone, Propoxyphene, Digoxin, Progesterone, Meperidine, Sotalol, Dextromethorphan, Anileridine, Diphenoxylate, Levomethadyl Acetate, Levorphanol, Methadyl Acetate, Oxymorphone, Remifentanil, Sufentanil, Apomorphine, Malathion, Buprenorphine, Pentazocine, Magnesium Sulfate, Thiopental, Hydrocortisone, Iron Dextran, Estradiol, Perphenazine, Fentanyl, Propofol, Naloxone, Estramustine, Finasteride, Paclitaxel, Dexamethasone, Acamprosate, Allopurinol, Aminocaproic Acid, Amoxapine, Bambuterol, Colchicine, Dasatinib, Diazepam, Ethopropazine, Haloperidol, Ketamine, L-Glutamic Acid, Loperamide, L-Phenylalanine, Orphenadrine, Oxprenolol, Raloxifene, Rasagiline, Trandolapril, Tranexamic Acid, Acebutolol, Acenocoumarol, Acetaminophen, Acetazolamide, Acetophenazine, Adenosine, Adinazolam, Alfuzosin, Almitrine, Alosetron, Alprazolam, Alprenolol, Alteplase, Aluminium, Amantadine, Amiloride, Aminolevulinic acid, Amiodarone, Amitriptyline, Amphetamine, Anakinra, Anisotropine Methylbromide, Aprepitant, Aprindine, Argatroban, Aripiprazole, Atenolol, Atomoxetine, Azacitidine, Benazepril, Benzphetamine, Benzquinamide, Benzthiazide, Benztropine, Bepridil, Betaxolol, Bethanechol, Bethanidine, Bevantolol, Bezafibrate, Biperiden, Bisoprolol, Brimonidine, Brinzolamide, Bromocriptine, Budesonide, Bumetanide, Bupivacaine, Bupropion, Buspirone, Butabarbital, Butalbital, Butethal, Cabergoline, Candesartan, Capecitabine, Carbamazepine, Carbetocin, Carbidopa, Carbinoxamine, Carmustine, Carphenazine, Carvedilol, Ceftriaxone, Celecoxib, Chlordiazepoxide, Chlormerodrin, Chlormezanone, Chloroquine, Chlorpromazine, Chlorprothixene, Chlorthalidone, Chlorzoxazone, Cholecalciferol, Ciclopirox, Cimetidine, Cinolazepam, Citalopram, Cladribine, Clenbuterol, Clobazam, Clomifene, Clomipramine, Clonazepam, Clonidine, Clorazepate, Clotiazepam, Clozapine, Corticotropin, Creatine, Cyclobenzaprine, Cyclopentolate, Cycrimine, Cysteamine, Cytarabine, Dacarbazine, Dapiprazole, Darbepoetin alfa, Debrisoquin, Decitabine, Desflurane, Desipramine, Dexfenfluramine, Dexmedetomidine, Dexrazoxane, Dextroamphetamine, Diazoxide, Diethylpropion, Diflunisal, Digitoxin, Dihydroergotamine, Divalproex sodium, Dobutamine, Docetaxel, Donepezil, Doxepin, Doxorubicin, Doxylamine, Droperidol, Duloxetine, Echothiophate Iodide, Eletriptan, Entacapone, Ephedrine, Epoetin alfa, Eprosartan, Ergoloid mesylate, Ergotamine, Erlotinib, Escitalopram, Esomeprazole, Estazolam, Estrone, Eszopiclone, Etanercept, Ethinamate, Ethosuximide, Ethotoin, Etomidate, Etoricoxib, Exenatide, Famotidine, Felbamate, Fencamfamine, Flavoxate, Floxuridine, Fluconazole, Fludrocortisone, Flumazenil, Flunarizine, Flunitrazepam, Fluoxetine, Flupenthixol, Fluphenazine, Flurazepam, Flurbiprofen, Fluspirilene, Flutamide, Fluvastatin, Fluvoxamine, Fosfomycin, Fosphenyloin, Frovatriptan, Gabapentin, Galantamine, Gefitinib, Ginkgo biloba, Ginseng, Glimepiride, Goserelin, Granisetron, Guanabenz, Guanethidine, Halazepam, Hexachlorophene, Hexobarbital, Homatropine Methylbromide, Hydralazine, Hyoscyamine, Ibuprofen, Idarubicin, Ifosfamide, Imipramine, Indomethacin, Insulin Lyspro recombinant, Insulin recombinant, Insulin, porcine, Interferon alfa-2a, Irbesartan, Isocarboxazid, Isofluorophate, Isoproterenol, Isosorbide Dinitrate, Isotretinoin, Ketoprofen, Ketorolac, Labetalol, Lamotrigine, Lansoprazole, Lapatinib, Latanoprost, Lenalidomide, Letrozole, Leucovorin, Leuprolide, Levallorphan, Levetiracetam, Levobunolol, Levobupivacaine, Levodopa, Liothyronine, Lipoic Acid, Lisinopril, Lisuride, Lithium, Lofexidine, Losartan, Lovastatin, Loxapine, L-Tyrosine, Lucanthone, Lutropin alfa, L-Valine, Maprotiline, Maraviroc, Marinol, Mazindol, Mecamylamine, Mecasermin, Mefenamic acid, Mefloquine, Megestrol, Melatonin, Meloxicam, Memantine, Mephenyloin, Mepivacaine, Meprobamate, Mercaptopurine, Mesoridazine, Metformin, Metharbital, Methazolamide, Methohexital, Methotrexate, Methotrimeprazine, Methyldopa, Methylphenidate, Methylphenobarbital, Methylprednisolone, Methyprylon, Methysergide, Metixene, Metoclopramide, Metoprolol, Metyrosine, Mexiletine, Mianserin, Midazolam, Mifepristone, Miglitol, Miglustat, Milnacipran, Minaprine, Mirtazapine, Modafinil, Molindone, Moricizine, Mycophenolate mofetil, Nabilone, Nadolol, Naltrexone, Nandrolone, Naproxen, Naratriptan, Natalizumab, Nefazodone, Nesiritide, Nicardipine, Nicergoline, Nicotine, Nisoldipine, Nitrazepam, Nitric Oxide, Nortriptyline, Olanzapine, Olmesartan, Omeprazole, Ondansetron, Orlistat, Ouabain, Oxazepam, Oxcarbazepine, Oxybutynin, Paliperidone, Palonosetron, Paramethadione, Paroxetine, Pemetrexed, Pentobarbital, Pentostatin, Pergolide, Perindopril, Phenelzine, Phenmetrazine, Phenobarbital, Phenoxybenzamine, Phentermine, Phenylbutazone, Phenylpropanolamine, Phenyloin, Phosphatidylserine, Physostigmine, Picrotoxin, Pilocarpine, Pimozide, Pindolol, Pirbuterol, Piroxicam, Pramipexole, Prazepam, Prednisone, Pregabalin, Prilocalne, Primidone, Procainamide, Prochlorperazine, Procyclidine, Progabide, Promazine, Propericiazine, Propiomazine, Propranolol, Protriptyline, Pseudoephedrine, Pyridoxal, Quazepam, Quetiapine, Quinestrol, Quinidine, Ramelteon, Ramipril, Ranitidine, Ranolazine, Reboxetine, Remoxipride, Reserpine, Ridogrel, Riluzole, Rimonabant, Risperidone, Ritodrine, Rituximab, Rivastigmine, Rizatriptan, Ropinirole, Ropivacaine, S-Adenosylmethionine, Salbutamol, Salicyclic acid, Salmeterol, Salmon Calcitonin, Salsalate, Scopolamine, Secobarbital, Secretin, Selegiline, Sermorelin, Sertindole, Sertraline, Sibutramine, Simvastatin, Sirolimus, Sitagliptin, Sodium lauryl sulfate, Solifenacin, Somatropin recombinant, Sorafenib, Spirapril, Streptokinase, Sulfasalazine, Sulpiride, Sumatriptan, Sunitinib, Suramin, Tacrine, Talbutal, Tamibarotene, Tamoxifen, Telmisartan, Temazepam, Tetrabenazine, Tetrahydrobiopterin, Thalidomide, Thiabendazole, Thiamine, Thiethylperazine, Thioguanine, Thioridazine, Tiagabine, Tizanidine, Tocamide, Tolbutamide, Tolcapone, Tolmetin, Topiramate, Torasemide, Tranylcypromine, Trazodone, Triamterene, Triazolam, Trifluoperazine, Triflupromazine, Trihexyphenidyl, Trimethadione, Trimetrexate, Trimipramine, Tropicamide, Valproic Acid, Valrubicin, Valsartan, Vapreotide, Vasopressin, Venlafaxine, Vigabatrin, Vinblastine, Vindesine, Voriconazole, Yohimbine, Zaleplon, Ziprasidone, Zolmitriptan, Zolpidem, Zonisamide, Zopiclone, Zuclopenthixol, Diminazene Aceturate, Milnacipran, Amlodipine, Aranidipine, Azelnidipine, Barnidipine, Cilnidipine, Clevidipine, Efonidipine, Lacidipine, Lercanidipine, Manidipine, Nifedipine, Nilvadipine, Nimodipine, Nitrendipine, Nitrepin, Pranidipine, Diltiazem, Mibefradil, Clodronate, Etidronate, Tiludronate, Pamidronate, Neridronate, Olpadronate, Ibandronate, Risedronate, and Zoledronate, useful for the treatment of neurodegenerative disorders.

Particularly advantageous embodiments of the combinations of this invention are combinations of two or more of riluzol, bepridil, diazoxide, thiamine, methylsergide, minaprine, alendronate, miconazole, melatonin, docetaxel, tamibarotene, ridogrel and diminazene aceturate, in amounts that are therapeutically effective for treating the neurodegenerative disorders described herein in a mammal, particularly in a mammal suffering from a neurodegenerative disease, and specifically in a human suffering from Alzheimer's Disease and associated dementias.

Particularly advantageous embodiments are the combinations of two or more of the compounds ridogrel, diminazene aceturate, riluzol, bepridil, docetaxel and alendronate, in amounts that are therapeutically effective for treating the neurodegenerative disorders described herein in a mammal, particularly in a mammal suffering from a neurodegenerative disease, and particularly in a human suffering from Alzheimer's Disease and associated dementias.

The invention relates further to treatment or prevention methods comprising drug-drug combinations and to kits containing drug-drug combinations.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1. TPMS technology comprises the principal steps of (1) Creating a map (2) Developing mathematical models, and (3) data analysis and experimental checking.

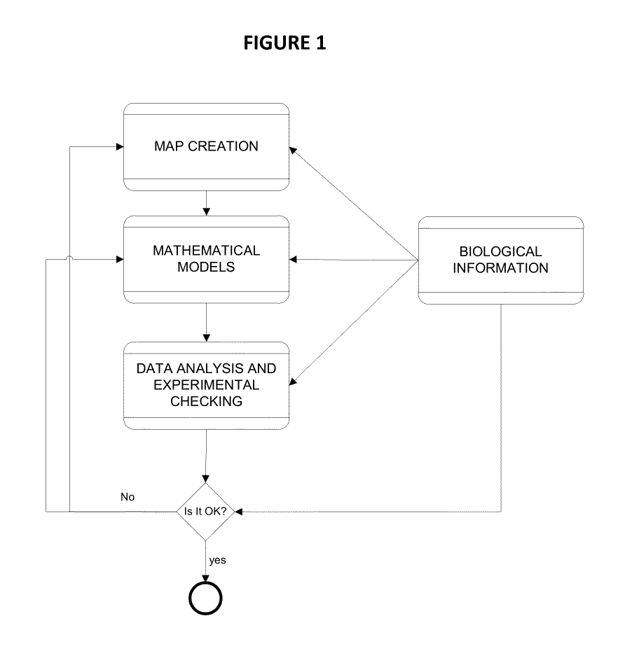

FIG. 2. Effect of Lansoprazole on amyloid pathology. Lansoprazole significantly increase β-amyloid (Aβ) 1-42 the more fibrillogenic form of Aβ, and reduces Aβ1-40/Aβ1-42 ratio. Data are mean±SEM values of 4 independent experiments (* p<0.05, ** p<0.01, *** p<0.001).

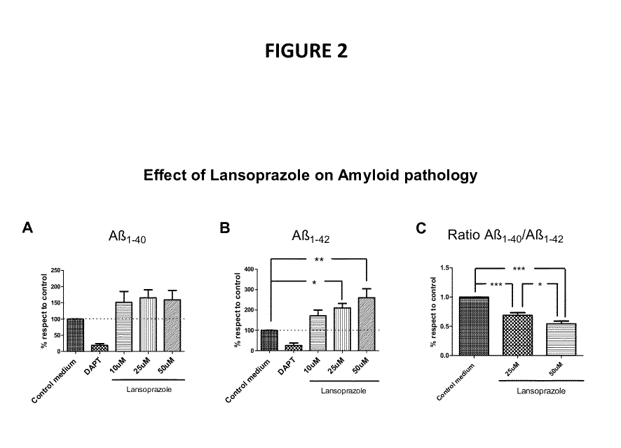

FIG. 3. Effect of Ridogrel on neuronal dysfunction and cell death motive. Ridogrel have shown a dose-dependent inhibition of AChE. Eserine 10 uM is used as positive control.

FIG. 4. Effect of Diminazene Aceturate on neuronal dysfunction and cell death motive. Diminazene Aceturate has shown a dose-dependent inhibition of AChE. Eserine 10 uM is used as positive control.

FIG. 5. Effect of Docetaxel on TAU pathology. Docetaxel significantly reduces pTAU/TAU ration on Docetaxel treated cells. Data are mean±SEM values of 3 independent experiments (* p<0.05).

FIG. 6. Effect of Bepridil+Riluzole on Memory. MWM probe trial for time spent in each quadrant of the pool (A) and for the ratio of time in the opposite quadrant respect to target quadrant (one containing the platform during training) (B). The ratio of time spent in the opposite quadrant compared time spent in the target quadrant revealed a 32.7% improvement of treated animal versus untreated animals (* p<0.05)

FIG. 7. Effect of Bepridil+Alendronate on Memory. Tracks of mice in the Morris water maze test on MWM probe trial (A) and ratio of time in the opposite quadrant respect to target quadrant (one containing the platform during training) (B). The ratio of time spent in the opposite quadrant compared time spent in the target quadrant revealed a 100% improvement of treated animal versus untreated animals. Anova test (* p<0.05).

FIG. 8. Synergistic effect of Bepridil+Alendronate on Memory. MWM probe trial for time spent in each quadrant (A) and for time spent in the area of influence around platform (B). Bepridil+Alendronate group display higher occupancy of the target quadrant (one containing the platform during training) compared to other mice groups. ANOVA test (* p<0.05).

### DESCRIPTION OF THE EMBODIMENTS

Reference will now be made in detail to the present embodiments (exemplary embodiments) of the invention, an example(s) of which is (are) illustrated in the accompanying drawings. Wherever possible, the same reference numbers will be used throughout the drawings to refer to the same or like parts.

Disclosed Drug-Drug Combinations

TPMS technology (Anaxomics Biotech SL, Barcelona, Spain) was used to discover new drug-drug combinations useful to treat neurodegenerative diseases.

Neurodegenerative Diseases where first characterized in four pathophysiological motives; Amyloid pathology, Tau pathology, Oxidative Stress and Neuronal dysfunction and death. The pathophysiological motives were characterized at protein level. The key proteins of each motive were identified and used as seed nodes to construct the Neurodegenerative Diseases biological Map. The map included 16255 proteins and its protein-to-protein interactions.

A mathematical model was developed that explained the behavior of the biological map in a mammal, especially in a human. The model related drug targets of known drugs identified as individual proteins, with the proteins related with the clinical phenotypes of relevance, mainly safety effects and mechanisms of action of neurological diseases. The model was restricted by network topology, i.e., by the described protein-to-protein interactions. A mechanistic model with sufficient accuracy and generalization power was generated and refined.

Drugs listed in DrugBank database (version January 2010), not used to train or develop the model, where then checked with the model. Drugs were tested in pairs of two drugs, to check if the effect of the drugs on the biological network and mathematical model could modulate the neurodegenerative diseases characterized as pathophysiological motives. More than 1,000,000 drug-drug combinations were computationally tested with the model.

A final group of drug combinations was obtained. High TPMS scores obtained by drug combinations with high individual prediction degree (prediction value equal or higher than 0.02), and high additive or synergistic degree by the highest single agent (HAS) model, were obtained for any combinations of at least two compounds as described in TABLE 1. Each of the drugs showed a specific score for each one of the four pathophysiological mechanisms or motives of neurodegenerative diseases described above: Amyloid pathology, Tau pathology, Oxidative Stress and Neuronal dysfunction and death. For each one of the drugs and the corresponding combinations as described in Table 1, the TPMS score is high for at least one of such mechanisms or motives

TABLE 1
List of drugs that when combined in combinations of at least two
compounds, score positive for having additive or synergistic effects for
treating neurological diseases, specifically neurodegenerative diseases.

| | | |
|---|---|---|
| L-Tyrosine | Phentermine | Tetrahydrobiopterin |
| Sermorelin | Enfuvirtide | Vidarabine |
| Oseltamivir | Cycloserine | Sulfisoxazole |
| Latanoprost | Ertapenem | Vindesine |
| Buclizine | Chlorzoxazone | Mefloquine |
| Miglustat | Trifluridine | Trimethoprim |
| Marinol | Divalproex sodium | Olopatadine |
| Gonadorelin | Thiabendazole | Histamine Phosphate |
| Acetohydroxamic Acid | Aprepitant | Amodiaquine |
| Paramethadione | Hexachlorophene | Fosfomycin |
| Cyclobenzaprine | Felbamate | Vitamin E |
| L-Proline | Ethopropazine | Streptokinase |
| Ethchlorvynol | Desflurane | Methoxyflurane |
| Salmon Calcitonin | Echothiophate Iodide | Tropicamide |
| Anakinra | Dofetilide | Lidocaine |
| Etomidate | Procyclidine | Picrotoxin |
| Gemcitabine | Nabilone | Pefloxacin |
| Chlormerodrin | Cimetidine | Clopidogrel |
| Amlexanox | Phenmetrazine | Brinzolamide |
| Progabide | Dipyridamole | Ramelteon |
| L-Glutamine | L-Aspartic Acid | L-Methionine |
| Adenosine triphosphate | Alteplase | Malathion |
| Chloroprocaine | Acamprosate | Azelaic Acid |
| Rifabutin | Riluzole | Ethanol |
| Simvastatin | Estrone | Glycine |
| Dextromethorphan | Estramustine | Orphenadrine |
| Methadone | Meperidine | Isoflurane |
| Bepridil | Adenosine monophosphate | Dasatinib |
| Natalizumab | NADH | Menadione |
| Sorafenib | Diethylstilbestrol | Flumethasone Pivalate |
| Dimethyl sulfoxide | Naloxone | Ketamine |
| Urokinase | Hyaluronidase | Tetrahydrofolic acid |
| S-Adenosylmethionine | L-Phenylalanine | L-Tryptophan |
| L-Isoleucine | Apomorphine | Tranexamic Acid |
| Triflupromazine | Etanercept | Palivizumab |
| Choline | Ibutilide | Chlorpromazine |
| Cyclothiazide | Bacitracin | Iron Dextran |
| Biotin | Sulindac | Minocycline |
| Clomipramine | L-Leucine | Haloperidol |
| Atorvastatin | Donepezil | Letrozole |
| Glutathione | L-Citrulline | Fludarabine |
| Arsenic trioxide | Pimozide | Insulin Lyspro recombinant |
| Phenoxybenzamine | Acetic Acid | Pyruvic acid |
| Succinic acid | Pegademase bovine | Human Serum Albumin |
| L-Histidine | L-Threonine | Amphetamine |
| L-Valine | Fluvoxamine | Tramadol |
| Esomeprazole | Lorazepam | Topiramate |
| Nalbuphine | Amitriptyline | Mirtazapine |
| Meprobamate | Montelukast | Thiamine |
| Methysergide | Dihydroergotamine | Aminocaproic Acid |
| Mephenytoin | Naltrexone | Pemetrexed |
| Lisuride | Magnesium Sulfate | Labetalol |
| gamma-Homolinolenic acid | Calcitriol | Cladribine |
| Benztropine | Digoxin | Alosetron |
| Quinine | Lisinopril | Thiopental |
| Nicardipine | Cinnarizine | Mycophenolate mofetil |
| Tranylcypromine | Hexobarbital | Atovaquone |
| Progesterone | Triamterene | Hydrocortisone |
| Atropine | Cetuximab | Alemtuzumab |
| L-Arginine | Spermine | L-Serine |
| Bethanidine | Liothyronine | Clozapine |
| Levothyroxine | Trazodone | Verapamil |
| Imatinib | Tretinoin | Cocaine |
| Alfentanil | Minaprine | Ketoprofen |
| Acetazolamide | Chlorprothixene | Vitamin C |
| L-Ornithine | Creatine | L-Cysteine |
| Icosapent | L-Alanine | Vitamin A |
| Hydroxocobalamin | Olanzapine | Thiethylperazine |
| Amiloride | Diazoxide | Tamoxifen |
| Estradiol | Felodipine | Phenelzine |
| Ranolazine | L-Lysine | Xanthophyll |
| Proguanil | Lisdexamfetamine | Carbidopa |
| Dapsone | Fluvastatin | Valproic Acid |
| Acarbose | Bupivacaine | Alfuzosin |
| Erlotinib | Metformin | Lansoprazole |
| Pyrazinamide | Terfenadine | Amoxapine |
| Framycetin | Cefdinir | Metyrosine |
| Sotalol | Astemizole | Roxithromycin |
| Pentostatin | Daunorubicin | Clofarabine |
| Oxazepam | Levamisole | Iloprost |
| Gatifloxacin | Rifampin | Amiodarone |
| Hydralazine | Mecasermin | Caffeine |
| Oxprenolol | Darbepoetin alfa | Secretin |
| Alpha-Linolenic Acid | Riboflavin | Pyridoxal |
| Aspartame | Cholecalciferol | Pravastatin |
| Candesartan | Acebutolol | Fluconazole |
| Enoxacin | Glimepiride | Nisoldipine |
| Atomoxetine | Butabarbital | Alclometasone |
| Acetaminophen | Pramipexole | Isradipine |
| Phenytoin | Venlafaxine | Flavoxate |
| Nitrofurazone | Thioguanine | Acetophenazine |
| Alprazolam | Dexrazoxane | Amlodipine |
| Anisotropine Methylbromide | Duloxetine | Clorazepate |
| Prochlorperazine | Cyproheptadine | Chloramphenicol |
| Cefalotin | Imipramine | Celecoxib |
| Brimonidine | Buspirone | Guanidine |
| Bupropion | Adenosine | Mitotane |
| Paroxetine | Nedocromil | Ciprofloxacin |
| Nortriptyline | Fluorouracil | Risperidone |
| Lamotrigine | Chloroquine | Thioridazine |
| Warfarin | Modafinil | Epinastine |
| Methotrexate | Propranolol | Niacin |
| Phentolamine | Clonidine | Pindolol |
| Mycophenolic acid | Trifluoperazine | Granisetron |
| Topotecan | Probenecid | Tiagabine |
| Quinidine | Zonisamide | Ezetimibe |
| Mercaptopurine | Procainamide | Selegiline |
| Carphenazine | Amantadine | Pseudoephedrine |
| Aminolevulinic acid | Cytarabine | Perindopril |
| Penicillamine | Azacitidine | Sulfasalazine |
| Gentamicin | Mesoridazine | Maprotiline |
| Gabapentin | Benzphetamine | Salbutamol |
| Alprenolol | Phenylbutazone | Fentanyl |
| Flupenthixol | Aspirin | Acenocoumarol |
| Balsalazide | Propofol | Levodopa |
| Aripiprazole | Cefazolin | Cefonicid |
| Ouabain | Itraconazole | Hesperetin |
| Isocarboxazid | Flucytosine | Capecitabine |
| Cyclosporine | Cyanocobalamin | L-Cystine |
| Nicotine | Rizatriptan | Benzquinamide |
| Ergotamine | Alendronate | Galantamine |
| Bumetanide | Quinacrine | Potassium Chloride |
| Ziprasidone | Promazine | Glibenclamide |
| Flunisolide | Mifepristone | Vinblastine |
| Isotretinoin | Fenofibrate | Chlorpheniramine |
| Cloxacillin | Raloxifene | Lithium |
| Lapatinib | Isoproterenol | Clonazepam |
| Paclitaxel | Rasagiline | Folic Acid |
| Trandolapril | Bethanechol | Floxuridine |
| Ethosuximide | Miglitol | Ribavirin |
| Tetracycline | Irinotecan | Dacarbazine |
| Fomepizole | Atazanavir | Amifostine |
| Halofantrine | Ciclopirox | Levetiracetam |
| Exenatide | L-Carnitine | Diazepam |
| Cyclizine | Tacrolimus | Thalidomide |
| Sirolimus | Goserelin | Chlorthalidone |
| Adinazolam | Cabergoline | Theophylline |
| Clotrimazole | Entacapone | Zanamivir |
| Cysteamine | Aciclovir | Biperiden |
| Isoniazid | Disulfiram | Enprofylline |
| Levocabastine | Sitagliptin | Nitrendipine |
| Sunitinib | Ergoloid mesylate | Praziquantel |
| Sulfinpyrazone | Cefadroxil | Ceftriaxone |
| Carbetocin | Bezafibrate | Levallorphan |
| Dexfenfluramine | Ridogrel | Argatroban |
| Carmustine | Isosorbide Dinitrate | Allopurinol |
| Diphenoxylate | Nitroglycerin | Piroxicam |
| Corticotropin | Ginkgo biloba | Ginseng |
| Halothane | Aluminium | Vorinostat |
| Heparin | Bromocriptine | Carvedilol |
| Doxepin | Orlistat | Saquinavir |
| Flunitrazepam | Digitoxin | Cefepime |
| Trimetrexate | Fencamfamine | Melatonin |
| Danazol | Pranlukast | Promethazine |
| Phosphatidylserine | Colchicine | Tamibarotene |
| Aprindine | Sertindole | Dexamethasone |
| Fluphenazine | Miconazole | Nefazodone |
| Desipramine | Quetiapine | Glucosamine |
| Dextroamphetamine | Clenbuterol | Lucanthone |
| Mianserin | Suramin | Irbesartan |
| Finasteride | Methotrimeprazine | Perphenazine |
| Flunarizine | Amrinone | Maraviroc |
| Sertraline | Metoclopramide | Nesiritide |
| Budesonide | Estriol | Docetaxel |
| Diminazene aceturate | Milnacipran | Amlodipine |
| Aranidipine | Azelnidipine | Barnidipine |
| Cilnidipine | Clevidipine | Efonidipine |
| Lacidipine | Lercanidipine | Manidipine |
| Nifedipine | Nilvadipine | Nimodipine |
| Nitrendipine | Nitrepin | Pranidipine |
| Diltiazem | Mibefradil | Clodronate |
| Etidronate | Tiludronate | Pamidronate |
| Neridronate | Olpadronate | Ibandronate |
| Risedronate | Zoledronate | |

The drug-drug combinations thus obtained are unexpected and surprising because the combinations described herein have not been described previously as useful for the treatment of neurodegenerative diseases.

The predictive values obtained are related with the mechanism of action of the drugs on the neurodegenerative diseases biological processes. Thus, particularly advantageous embodiments of the combinations of this invention are combinations of at least one calcium channel blocker and at least one bisphosphonate.

Calcium channel blockers can be of the class Dihydropyridine (Amlodipine, Aranidipine, Azelnidipine, Barnidipine, Benidipine, Cilnidipine, Clevidipine, Isradipine, Efonidipine, Felodipine, Lacidipine, Lercanidipine, Manidipine, Nicardipine, Nifedipine, Nilvadipine, Nimodipine, Nisoldipine, Nitrendipine, Nitrepin, and Pranidipine), of the class Phenilalkylamine (Verapamil), of the class Benzothiazepine (Diltiazem), and of the class of Non Selective calcium channel blockers (Mibefradil, Bepridil, Fluspirilene, and Fendiline).

Bisphosphonates can be of the class of Non-nitrogenous bisphosphonates (Etidronate, Clodronate, Tiludronate), or of the class of Nitrogenous bisphosphonates (Pamidronate, Neridronate, Olpadronate, Alendronate, Ibandronate, Risedronate, Zoledronate).

Particularly advantageous embodiments of the combinations of this invention are combinations of two or more of riluzol, bepridil, diazoxide, thiamine, methylsergide, minaprine, alendronate, miconazole, melatonin, docetaxel, tamibarotene, ridogrel and diminazene aceturate, in amounts that are therapeutically effective for treating the neurodegenerative disorders described herein in a mammal, particularly in a mammal suffering from a neurodegenerative disease, and specifically in a human suffering from Alzheimer's Disease and associated dementias.

Particularly advantageous embodiments are the combinations of two or more of the compounds ridogrel, diminazene aceturate, riluzol, bepridil, docetaxel and alendronate, in amounts that are therapeutically effective for treating the neurodegenerative disorders described herein in a mammal, particularly in a mammal suffering from a neurodegenerative disease, and particularly in a human suffering from Alzheimer's Disease and associated dementias.

The compositions of this invention are useful for the treatment of central nervous system diseases, in particular neurodegenerative diseases, and more particularly for the treatment of neurological disorders associated with neurodegeneration including but not limited to Parkinson Disease, Tauopathies, Alzheimer's Disease, Diffuse Neurofibrillary Tangles with Calcification, Supranuclear Palsy, Progressive, TDP-43 Proteinopathies, Amyotrophic Lateral Sclerosis, Frontotemporal Lobar Degeneration, Lewy Body Disease, AIDS Dementia Complex, Aphasia, Primary Progressive, Primary Progressive Nonfluent Aphasia, Dementia, Vascular, CADASIL, Dementia, Multi-Infarct, Diffuse Neurofibrillary Tangles with Calcification, Frontotemporal Lobar Degeneration, Frontotemporal Dementia, Primary Progressive Nonfluent Aphasia, Kluver-Bucy Syndrome, Pick's Disease, Motor Neuron Disease, Amyotrophic Lateral Sclerosis, Bulbar Palsy, Progressive, Muscular Atrophy, Spinal, Multiple System Atrophy, Olivopontocerebellar Atrophies, Shy-Drager Syndrome, Striatonigral Degeneration, Olivopontocerebellar Atrophies, Paraneoplastic Syndromes, Nervous System, Lambert-Eaton Myasthenic Syndrome, Limbic Encephalitis, Myelitis, Transverse, Opsoclonus-Myoclonus Syndrome, Paraneoplastic Cerebellar Degeneration, Paraneoplastic Polyneuropathy, Postpoliomyelitis Syndrome, Prion Diseases, Encephalopathy, Bovine Spongiform, Gerstmann-Straussler-Scheinker Disease, Insomnia, Fatal Familial, Kuru, Scrapie, Wasting Disease, Chronic, Creutzfeldt-Jakob Syndrome, Shy-Drager Syndrome, Subacute Combined Degeneration, Heredodegenerative Disorders, Nervous System, Alexander Disease, Amyloid Neuropathies, Familial, Bulbo-Spinal Atrophy, X-Linked, Canavan Disease, Cockayne Syndrome, Dystonia Musculorum Deformans, Gerstmann-Straussler-Scheinker Disease, Hepatolenticular Degeneration, Hereditary Central Nervous System Demyelinating Diseases, Hereditary Sensory and Autonomic Neuropathies, Hereditary Sensory and Motor Neuropathy, Huntington Disease, Lafora Disease, Lesch-Nyhan Syndrome, Menkes Kinky Hair Syndrome, Myotonia Congenita, Myotonic Dystrophy, Neurofibromatoses, Neuronal Ceroid-Lipofuscinoses, Optic Atrophies, Hereditary, Pantothenate Kinase-Associated Neurodegeneration, Rett Syndrome, Spinal Muscular Atrophies of Childhood, Spinocerebellar Degenerations, Tourette Syndrome, Tuberous Sclerosis, Unverricht-Lundborg Syndrome, and the similar, and more particularly for the treatment of Alzheimer's Disease and associated dementias.

Particularly advantageous embodiments are the combinations in which the different compounds in the combination are directed to different molecular causative motives of the disease. For example, if the neurodegenerative disease to be treated is Alzheimer's disease, causative motives can be Amyloid pathology, Tau pathology, Oxidative Stress and Neuronal dysfunction and death.

In a particular embodiment, the compositions described herein can be used to treat a neurological disease that involves one or more of the physiopathological processes Amyloid pathology, Tau pathology, Oxidative Stress and Neuronal dysfunction and death.

Particularly advantageous embodiments of the invention are the combinations of two compounds as described in Table 1, plus at least one or more drugs used to treat neurological diseases.

In a particular embodiment the compounds are used to prepare a medicine for treating a mammal in need thereof, in particular a human patient, wherein compounds are used in a dosage of 0.0001 mg/kg to 1000 mg/kg of body weight, in particular from 0.01 mg/kg to 100 mg/kg of body weight. In particular, the combination of riluzol+bepridil is used in a range of 0.001 mg/kg day to 152 mg/Kg day for riluzol and 0.05 mg/Kg day to 692 mg/Kg day for bepridil, the combination of alendronate+bepridil is used in a range of 0.001 mg/kg day to 150 mg/Kg day for alendronate and 0.05 mg/Kg day to 692 mg/Kg day for bepridil, and the combination of alendronate+docetaxel is used in a range of 0.001 mg/kg day to 150 mg/Kg day for alendronate and 0.001 mg/Kg day to 83 mg/Kg day for bepridil.

Structural and functional analogs of each of these compounds are known, and any of these analogs can be prepared by persons of ordinary skills in the art and used in the combinations of the invention, to the same extent as the parental compounds.

Metabolites of the compounds of the invention are also commonly known by persons skilled in the art. Many of these metabolites share one or more biological activities with the parent compounds and, accordingly, can also be used in the combinations of the invention, to the same extent as the parental compounds.

Pharmaceutical Compositions

This invention also provides pharmaceutical compositions that comprise compounds of this invention formulated together with one or more non-toxic pharmaceutically acceptable carriers. The pharmaceutical compositions may be specially formulated for oral administration in solid or liquid form, for parenteral injection, or for rectal administration. The invention particularly provides pharmaceutical compositions that comprise any combination of at least two or more compounds selected from the list of drugs included in TABLE 1, and in particular the combinations of two or more of riluzol, bepridil, diazoxide, thiamine, methylsergide, minaprine, alendronate, bepridil, miconazole, melatonin, docetaxel, tamibarotene, ridogrel and diminazene aceturate, and more particulary the combinations of two or more of the compounds ridogrel, diminazene aceturate, riluzol, bepridil, docetaxel and alendronate optionally formulated together with one or more non-toxic pharmaceutically acceptable carriers. The pharmaceutical compositions may be specially formulated for oral administration in solid or liquid form, for parenteral injection, or for rectal administration.

The term “pharmaceutically acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. This invention provides pharmaceutical compositions which comprise compounds of the invention formulated together with one or more non-toxic pharmaceutically acceptable carriers. The pharmaceutical compositions can be formulated for oral administration in solid or liquid form, for parenteral injection or for rectal administration.

The pharmaceutical compositions of this invention can be administered to humans (patients) and other mammals orally, rectally, parenterally, intracisternally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of this invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more pharmaceutically acceptable carriers as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert pharmaceutically acceptable carrier such as sodium citrate or calcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of this invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of this invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

The phrase “therapeutically effective amount” of the compound of this invention means a sufficient amount of the compound to treat neurological and/or neurodegenerative disorders, or to prevent the onset of neurological and/or neurodegenerative disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Actual dosage levels of active ingredients in the pharmaceutical compositions of this invention can be varied so as to obtain an amount of the active compound(s) which is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated.

A “pharmaceutically-acceptable derivative” denotes any salt, ester of a compound of this invention, or any other compound which upon administration to a patient is capable of providing (directly or indirectly) a compound of this invention, or a metabolite or residue thereof.

The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, [beta]-hydroxybutyric, salicylic, galactaric and galacturonic acid. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, aistidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention. When a basic group and an acid group are present in the same molecule, a compound of the invention may also form internal salts.

This invention contemplates pharmaceutically active metabolites formed by in vivo biotransformation of, any of the compounds included in any list of compounds previously disclosed.

Kits Containing Compositions

The invention is also directed to a method of administration of the combination. More particularly the active agents of the combination therapy are administered sequentially in either order or simultaneously. When the active agents are administered simultaneously, one skilled in the art will understand that the second agent can be administered some time after the first agent. The particular period of delay is dependent on the particular pharmacokinetic and formulation parameters of the active agent. The particular period of delay between the administration of the individual compounds in the combination can extend to days, hours, minutes or seconds.

The invention also relates to a kit, wherein the individual compounds of the combination are disposed in separate containers.

The invention also relates to a kit according to any of the foregoing, further comprising integrally thereto or as one or more separate documents, information pertaining to the contents or the kit and the use of the inhibitors.

As used in relation to the invention, the term “treating” or “treatment” and the like should be taken broadly. They should not be taken to imply that an animal is treated to total recovery. Accordingly, these terms include amelioration of the symptoms or severity of a particular condition or preventing or otherwise reducing the risk of further development of a particular condition.

The term “comprising” is meant to be open ended, including the indicated component but not excluding other elements.

The phrase “therapeutically-effective” is intended to qualify the amount of each agent, which will achieve the goal of improvement in disorder severity and the frequency of incidence over treatment of each agent by itself, while avoiding adverse side effects typically associated with alternative therapies.

It should be appreciated that methods of the invention may be applicable to various species of subjects, preferably mammals, more preferably humans.

As used herein, the compounds of the present invention include the pharmaceutically acceptable derivatives thereof.

Where the plural form is used for compounds, salts, and the like, this is taken to mean also a single compound, salt and the like.

Methods of Treatment

The invention thus provides a method for treating central nervous system diseases, in particular neurodegenerative diseases, and more particularly for the treatment of neurological disorders associated with neurodegeneration including but not limited to Parkinson Disease, Tauopathies, Alzheimer's Disease, Diffuse Neurofibrillary Tangles with Calcification, Supranuclear Palsy, Progressive, TDP-43 Proteinopathies, Amyotrophic Lateral Sclerosis, Frontotemporal Lobar Degeneration, Lewy Body Disease, AIDS Dementia Complex, Aphasia, Primary Progressive, Primary Progressive Nonfluent Aphasia, Dementia, Vascular, CADASIL, Dementia, Multi-Infarct, Diffuse Neurofibrillary Tangles with Calcification, Frontotemporal Lobar Degeneration, Frontotemporal Dementia, Primary Progressive Nonfluent Aphasia, Kluver-Bucy Syndrome, Pick's Disease, Motor Neuron Disease, Amyotrophic Lateral Sclerosis, Bulbar Palsy, Progressive, Muscular Atrophy, Spinal, Multiple System Atrophy, Olivopontocerebellar Atrophies, Shy-Drager Syndrome, Striatonigral Degeneration, Olivopontocerebellar Atrophies, Paraneoplastic Syndromes, Nervous System, Lambert-Eaton Myasthenic Syndrome, Limbic Encephalitis, Myelitis, Transverse, Opsoclonus-Myoclonus Syndrome, Paraneoplastic Cerebellar Degeneration, Paraneoplastic Polyneuropathy, Postpoliomyelitis Syndrome, Prion Diseases, Encephalopathy, Bovine Spongiform, Gerstmann-Straussler-Scheinker Disease, Insomnia, Fatal Familial, Kuru, Scrapie, Wasting Disease, Chronic, Creutzfeldt-Jakob Syndrome, Shy-Drager Syndrome, Subacute Combined Degeneration, Heredodegenerative Disorders, Nervous System, Alexander Disease, Amyloid Neuropathies, Familial, Bulbo-Spinal Atrophy, X-Linked, Canavan Disease, Cockayne Syndrome, Dystonia Musculorum Deformans, Gerstmann-Straussler-Scheinker Disease, Hepatolenticular Degeneration, Hereditary Central Nervous System Demyelinating Diseases, Hereditary Sensory and Autonomic Neuropathies, Hereditary Sensory and Motor Neuropathy, Huntington Disease, Lafora Disease, Lesch-Nyhan Syndrome, Menkes Kinky Hair Syndrome, Myotonia Congenita, Myotonic Dystrophy, Neurofibromatoses, Neuronal Ceroid-Lipofuscinoses, Optic Atrophies, Hereditary, Pantothenate Kinase-Associated Neurodegeneration, Rett Syndrome, Spinal Muscular Atrophies of Childhood, Spinocerebellar Degenerations, Tourette Syndrome, Tuberous Sclerosis, Unverricht-Lundborg Syndrome, and the similar, and more particularly for the treatment of Alzheimer's Disease and associated dementias, in a mammal in need thereof, particularly in a human patient, that includes the step of administering to the mammal, particularly to the human mammal, a therapeutically effective amount, particularly a synergistically effective amount of a pharmaceutical composition of a pharmaceutical combination comprising any combination of at least two or more of the drugs included in TABLE 1, and in particular the combinations of two or more of riluzol, bepridil, diazoxide, thiamine, methylsergide, minaprine, alendronate, miconazole, melatonin, docetaxel, tamibarotene, ridogrel and diminazene aceturate, and more particularly the combinations of two or more of the compounds ridogrel, diminazene aceturate, riluzol, bepridil, docetaxel and alendronate and at least one pharmaceutically acceptable carrier.

In another embodiment, the present combinations may also be used with other types of therapies for treating neurodegenerative diseases.

In another embodiment, the present combinations may also be used or administered in combination with other drugs for the treatment of neurodegenerative diseases or concomitant neurological diseases. As will be appreciated, the dose of a combination of the present invention to be administered, the period of administration, and the general administration regime may differ between subjects depending on such variables as the severity of symptoms, the type of neurodegenerative disease to be treated, the mode of administration chosen, type of composition, size of a unit dosage, kind of excipients, the age and/or general health of a subject, and other factors well known to those of ordinary skill in the art.

Administration may include a single daily dose or administration of a number of discrete divided doses as may be appropriate. An administration regime may also include administration of one or more of the active agents, or compositions comprising same, as described herein. The period of administration may be variable. It may occur for as long a period is desired.

Administration may include simultaneous administration of suitable agents or compositions or sequential administration of agents or compositions.

In a further embodiment, the compositions and methods described herein may be used prophylactically as a means to prevent the development and/or onset of neurodegenerative diseases and/or associated symptoms.

Specifically, in the case of Alzheimer Disease and related dementias, the compounds and methods described herein may be used to treat or to prevent early forms of cognitive impairment, memory loss or mild dementia. The compounds and methods described herein can be used also as memory protection treatments or preventive treatments.

The following examples further illustrate specific embodiments of the invention; however, the following illustrative examples should not be interpreted in any way to limit the extent of the invention.

### EXAMPLES

### Example 1

Alzheimer's disease (AD) was characterized in four pathophysiological motives; Amyloid pathology, Tau pathology, Oxidative Stress and Neuronal dysfunction and death at protein level. The key proteins of each motive are identified and used as seed nodes to construct the Alzheimer's disease Map. A mathematical model was developed to mechanistically reproduce the behavior of the biological map, and to be able to generalize to new predictions.

A final group of drug combinations was obtained. High TPMS scores obtained by drug combinations with high individual prediction degree (prediction value equal or higher than 0.02), and high additive or synergistic degree by the highest single agent (HAS) model, were obtained for any combinations of at least two compounds as described in TABLE 1. Each of the drugs showed a specific score for each one of the four pathophysiological mechanisms or motives of neurodegenerative diseases described above: Amyloid pathology, Tau pathology, Oxidative Stress and Neuronal dysfunction and death. For each one of the drugs and the corresponding combinations as described in Table 1, the TPMS score is high for at least one of such mechanisms or motives

### Example 2

Particularly high prediction and synergism values were obtained for the combination of two or more among riluzol, bepridil, diazoxide, thiamine, methylsergide, minaprine, alendronate, miconazole, melatonin, docetaxel, tamibarotene, ridogrel, milnacipran and diminazene aceturate (Table 2).

TABLE 2
Drug combinations with high TPMS score. TPMS score
obtained by high prediction degree, prediction value equal or higher
than 0.02 and high synergism degree.

| Combinations | | Predicted | | | |
|---|---|---|---|---|---|
| Drug A | Drug B | Combination | Drug A | Drug B | Synergism |
| Riluzole | Bepridil | 0.16 | 0.08 | 0.06 | + |
| Riluzole | Diazoxide | 0.14 | 0.08 | 0.02 | + |
| Thiamine | Riluzole | 0.12 | 0.04 | 0.08 | + |
| Methysergide | Riluzole | 0.12 | 0.04 | 0.08 | + |
| Alendronate | Felodipine | 0.10 | 0.04 | 0.06 | + |
| Alendronate | Bepridil | 0.10 | 0.04 | 0.06 | + |
| Alendronate | Miconazole | 0.10 | 0.04 | 0.08 | + |
| Alendronate | Melatonin | 0.10 | 0.04 | 0.08 | + |
| Alendronate | Docetaxel | 0.08 | 0.04 | 0.04 | + |
| Alendronate | Tamibarotene | 0.08 | 0.04 | 0.04 | + |
| Bepridil | Tamibarotene | 0.08 | 0.06 | 0.04 | + |
| Riluzole | Minaprine | 0.12 | 0.08 | 0.04 | + |
| Riluzole | Minaprine | 0.12 | 0.08 | 0.04 | + |
| Docetaxel | Diminazene | 0.22 | 0.18 | 0.14 | + |
| Ridogrel | Milnacipran | 0.06 | 0.02 | 0.04 | + |
| Norgestimate | Docetaxel | 0.26 | 0.16 | 0.18 | + |
| Loteprednol | Docetaxel | 0.24 | 0.18 | 0.18 | + |
| Etabonate | | | | | |
| Loteprednol | Diminazene | 0.22 | 0.18 | 0.14 | + |
| Etabonate | | | | | |

### Example 3

As per example combination of bepridil, a calcium channel blocker used to treat angina, with riluzole, a glutamate antagonist used as anticonvulsant and used to prolong the survival of patients with amyotrophic lateral sclerosis, has shown a hit rate of 98% and an prediction value of 0.16, which means a possibility of 84.81% to correctly predict its indication on AD pathology. Synergy between bepridil and riluzole has been predicted.

Therefore, the efficacy of the drug combination on memory was studied in a mice model of Alzheimer's disease (3×Tg-AD) overexpressing human PS1_{M146V}, tau_{P301L}, APP_{SWE} [16] using spatial reference learning and memory testing (Morris water maze test (MWM test)). Two groups of female 3×Tg-AD mice were administered bepridil+riluzole (n=14) or vehicle (n=11) starting at 4 months of age during 10 consecutive weeks. A group of age and gender-matched non-transgenic littermate controls (wild-type) received vehicle (n=11) on a similar timetable schedule. Drugs and vehicle (10% DMSO, 18% encapsin) were administered by oral gavage to 3×Tg-AD and wild-type mice at a daily dose of 50 mg/Kg bepridil and 15 mg/Kg riluzole.

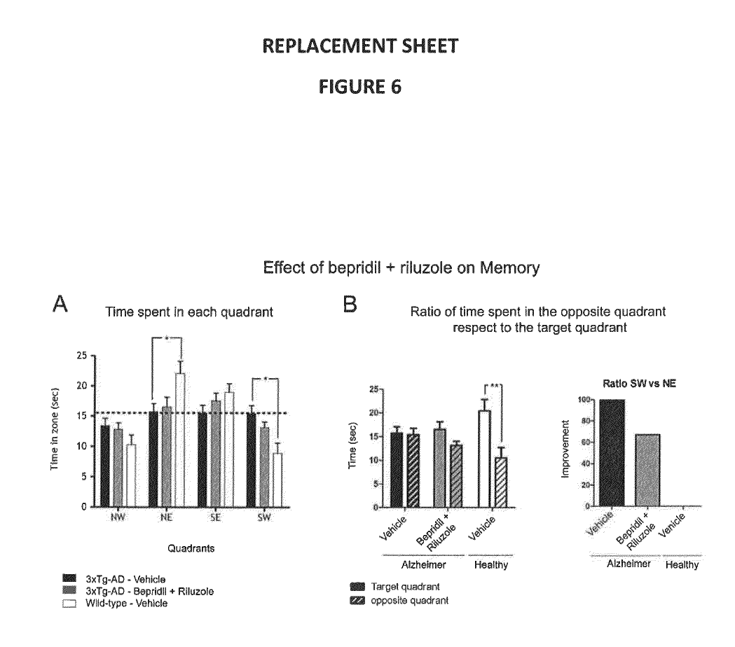

Wild-type mice and treated-3×Tg-AD mice display higher occupancy of the target quadrant (NE) compared to other quadrants (NE,SE,SW), whereas 3×Tg-AD transgenic mice spend less time searching the virtual target platform. The ratio of time spent in the opposite quadrant compared time spent in the target quadrant (one containing the platform during training) revealed a 32.7% improvement of treated animal versus untreated animals (FIG. 6).

### Example 4

As per example combination of alendronate, a bisphosphonate used to treat osteoporosis and Paget's disease and bepridil, a calcium channel blocker used to treat angina, has shown a hit rate of 98% and a prediction value of 0.1, which means a possibility of 81.12% to correctly predict its indication on AD pathology. Synergy between bepridil and alendronate has been predicted.

Therefore, the efficacy of the drug combination on memory was studied in a mice model of Alzheimer's disease (3×Tg-AD) overexpressing human PS1_{M146V}, tau_{P301L}, APP_{SWE [}16] using spatial reference learning and memory testing (MWT test). Two groups of female 3×Tg-AD mice were administered bepridil+alendronate (n=20) or vehicle (n=21) starting at 4.5 months of age during 10 consecutive months. A group of age and gender-matched non-transgenic littermate controls (wild-type) received vehicle (n=21) on a similar timetable schedule. Drugs and vehicle (10% DMSO, 18% encapsin) were administered by oral gavage to 3×Tg-AD and wild-type mice at a daily dose of 50 mg/Kg bepridil and 5 mg/Kg alendronate.

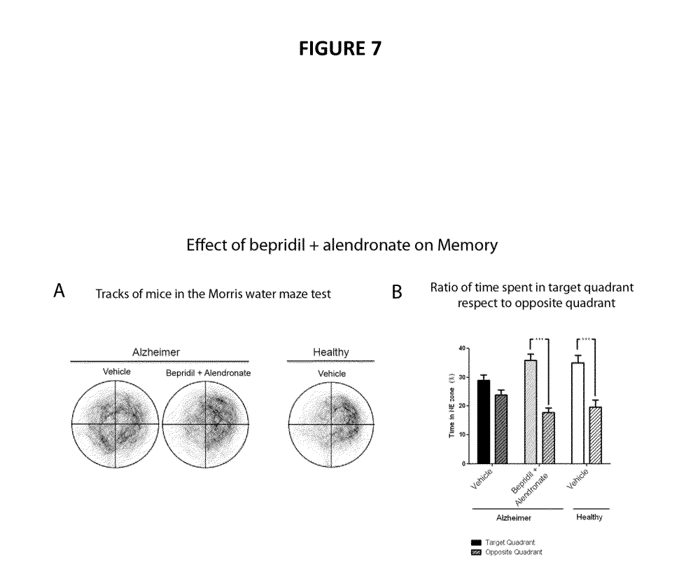

Wild-type mice and treated-3×Tg-AD mice display higher occupancy of the target quadrant (NE) compared to other quadrants (NE,SE,SW), whereas 3×Tg-AD transgenic mice spent less time searching the virtual target platform. The ratio of time spent in the opposite quadrant compared time spent in the target quadrant (one containing the platform during training) revealed a 100% improvement of treated animal compared to untreated animals (FIG. 7).

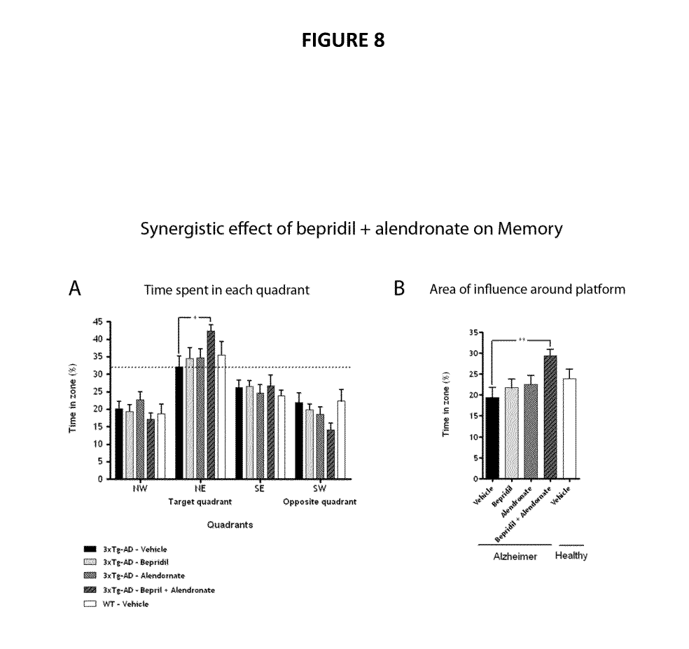

The synergistic effect of the drug combination on memory was studied in a mice model of Alzheimer's disease (3×Tg-AD) overexpressing human PS1_{M146V}, tau_{P301L}, APP_{SWE [}16] using spatial reference learning and memory testing (MWM test). Four groups of female 3×Tg-AD mice were administered bepridil (n=10), alendronate (n=10), bepridil+alendronate (n=10) or vehicle (n=10) starting at 4.5 months of age during 10 consecutive months. A group of age and gender-matched non-transgenic littermate controls (wild-type) received vehicle (n=10) on a similar timetable schedule. Drugs and vehicle (10% DMSO, 18% encapsin) were administered by oral gavage to 3×Tg-AD and wild-type mice at a daily dose of 50 mg/Kg bepridil and 5 mg/Kg alendronate. Synergism in terms of efficacy for treating neurodegenerative diseases has been observed with the combined administration of bepridil+alendronate (FIG. 8), as a more than additive effect was observed versus the administration of bepridil alone or alendronate alone.

### Example 5

As per example combination of alendronate, a bisphosphonate used to treat osteoporosis and Paget's disease with docetaxel, an anti-mitotic chemotherapy medication, has shown a hit rate of 98% and a prediction value of 0.082, which means a possibility of 79.53% to correctly predict its indication on AD pathology. Synergy between alendronate+docetaxel has been predicted.

### Example 6

Capacity of the TPMS technology to predict positive controls, i.e., drugs currently used or studied for the treatment of neurodegenerative diseases.

TPMS score was determinate using the predicted value of the currently drugs used to treat AD such as memantine, rivastigmine, donepezil and galantamine and also, drugs on AD clinical trials such as vitamin E, Melatonin, Estrone, Choline, Thiamine, Buspirone, Estradiol, Mifepristone, Minaprine, Flurbiprofen, Celecoxib, Dapsone, Valproic Acid, Lovastatin, Atorvastatin, Ginseng, Nicotinamide and Lithium. An equal or higher score of currently drugs tested for AD was obtained for Acamprosate, Rifabutin, Miconazole, Phenoxybenzamine, Fluphenazine, Aprindine, Riluzole, Isotretinoin, Bepridil, Perphenazine, Pimozide, Flunarizine, Felodipine, Alendronate, Flunisolide, Estriol, Quinestrol, Irbesartan, Budesonide, Docetaxel, Finasteride, Tamibarotene, Phentolamine, Isradipine, Aspartame, Adenine, Warfarin, Darbepoetin alfa, Nicergoline, Phenyloin, Cholecalciferol, Eletriptan, Sumatriptan, Clonidine, Bevantolol, Tocamide, Atomoxetine, Fluoxetine, Ropinirole, Mercaptopurine, Naratriptan, Sertraline, Metoclopramide, Nesiritide, Fenofibrate, Tacrolimus, Hydrocortisone, Estramustine, Sirolimus, Letrozole, Vinblastine, Ciprofloxacin, Gatifloxacin, Mephenyloin, Cytarabine, Probenecid, Urokinase, Hyaluronidase, L-Arginine, Cetuximab, L-Cysteine, Icosapent, Levothyroxine, Verapamil, Sulindac, Iloprost, Cocaine, Cefazolin, Insulin recombinant, Insulin Lyspro recombinant, Etanercept, Methysergide, Chlorpromazine, Debrisoquin, Thiethylperazine, Lipoic Acid, Fluvoxamine, Liothyronine, Amitriptyline, Clozapine, Mirtazapine, Trazodone, Risperidone, Lamotrigine, Thioridazine, Diflunisal, Flupenthixol, Levodopa, Ketoprofen, Nefazodone, Paliperidone, Sertindole, L-Citrulline, Malathion, Palivizumab, Trimethoprim, Amodiaquine, Ibutilide, Cyclothiazide, Framycetin, Astemizole, Roxithromycin, Daunorubicin, Clofarabine, Calcitriol, Quinine, Cinnarizine, Bacitracin, Mycophenolic acid, Pyrazinamide, Cefdinir, Biotin, Pegademase bovine, Alemtuzumab, Tetrahydrofolic acid, L-Lysine, Levofloxacin, Pyrimethamine, Beclomethasone, Chloramphenicol, Tetracycline, Irinotecan, L-Isoleucine, Acarbose, Topotecan, Flucytosine, Quinacrine, Gentamicin, Itraconazole, Atazanavir, Amifostine, Halofantrine, Glucosamine, Amrinone, Heparin, Saquinavir, Natalizumab, Phentermine, Tetrahydrobiopterin, Mefloquine, Magnesium Sulfate, Labetalol, Iron Dextran, Pindolol, Sotalol, Metyrosine, Benztropine, Meprobamate, Digoxin, Naltrexone, Lisuride, Thiopental, Nicardipine, Pseudoephedrine, Alprenolol, Levallorphan, Pemetrexed, Mycophenolate mofetil, Cladribine, Ranolazine, Secretin, L-Histidine, S-Adenosylmethionine, Methazolamide, Metformin, Adenosine, Nortriptyline, L-Tryptophan, L-Serine, Caffeine, Lorazepam, Carbidopa, Bupivacaine, Methylphenidate, Prochlorperazine, Paroxetine, Chloroquine, Loxapine, Idarubicin, Levobupivacaine, Methoxamine, Omeprazole, Ouabain, Selegiline, Dacarbazine, Quinidine, Zonisamide, Carphenazine, Amantadine, Maprotiline, Gabapentin, Salbutamol, Acetazolamide, Amiodarone, Diazoxide, Exenatide, Isocarboxazid, Dextroamphetamine, Clenbuterol, Trimetrexate, Azacitidine, Carvedilol, Fencamfamine, Suramin, Maraviroc, Mianserin, gamma-Homolinolenic acid, Cyclosporine, L-Glutamine, L-Aspartic Acid, Cyproheptadine, L-Methionine, Adenosine triphosphate, Diethylstilbestrol, Nitrofurazone, Nitroglycerin, Balsalazide, Aspirin, Danazol, Pranlukast, Vorinostat, Doxylamine, Cyclobenzaprine, Lisinopril, Benzphetamine, Phenylbutazone, Progesterone, Tiagabine, Carmustine, Simvastatin, Piroxicam, Tamoxifen, Olmesartan, Lofexidine, Rizatriptan, Glimepiride, Naloxone, Ridogrel, Dexamethasone, Ciclopirox, Bezafibrate, Paclitaxel, Methoxyflurane, Epinastine, Vitamin C, Glutathione, L-Alanine, Pravastatin, Hydroxocobalamin, Imatinib, Minocycline, Halothane, Glibenclamide, Vasopressin, Dihydroergotamine, Amiloride, Ergotamine, Triflupromazine, Zolpidem, Venlafaxine, Bumetanide, Dexfenfluramine, Bromocriptine, Methotrimeprazine, Clomipramine, Clonazepam. Among them, combinations of drugs that modulate the target candidates so identified on different pathophysiological motive are particularly preferred embodiment of this invention.

Among them, the higher TPMS score was obtained for Acamprosate, Miconazole, Aprindine, Riluzole, Bepridil, Flunarizine, Felodipine, Alendronate, Flunisolide, Quinestrol, Irbesartan, Docetaxel, Finasteride, Tamibarotene, Isradipine, Aspartame, Warfarin, Darbepoetin alfa, Nicergoline, Phenyloin, Cholecalciferol, Hydralazine and Eletriptan (Table 3).

TABLE 3: Drug candidates with high TPMS score. TPMS score is determinate using the predicted value of the currently drugs used to treat AD and drugs on AD clinical trials

TABLE 3

| | | Current and |
|---|---|---|
| | Predicted | clinical trial |
| Drug | Value | AD drugs |
| Memantine | 0.16 | + |
| Phosphatidylserine | 0.12 | + |
| Acamprosate | 0.12 | |
| Vitamin E | 0.10 | + |
| Miconazole | 0.08 | |
| Melatonin | 0.08 | + |
| Aprindine | 0.08 | |
| Riluzole | 0.08 | |
| Rivastigmine | 0.08 | + |
| Galantamine | 0.08 | + |
| Bepridil | 0.06 | |
| Flunarizine | 0.06 | |
| Felodipine | 0.06 | |
| Estrone | 0.06 | + |
| Donepezil | 0.06 | + |
| Choline | 0.06 | + |
| Alendronate | 0.04 | |
| Flunisolide | 0.04 | |
| Quinestrol | 0.04 | |
| Irbesartan | 0.04 | |
| Docetaxel | 0.04 | |
| Finasteride | 0.04 | |
| Tamibarotene | 0.04 | |
| Isradipine | 0.04 | |
| Aspartame | 0.04 | |
| Warfarin | 0.04 | |
| Darbepoetin alfa | 0.04 | |
| Nicergoline | 0.04 | |
| Phenytoin | 0.04 | |
| Cholecalciferol | 0.04 | |
| Hydralazine | 0.04 | |
| Eletriptan | 0.04 | |
| Buspirone | 0.04 | + |
| Thiamine | 0.04 | + |
| Estradiol | 0.04 | + |
| Mifepristone | 0.04 | + |
| Flurbiprofen | 0.04 | + |
| Celecoxib | 0.04 | + |

### Example 7

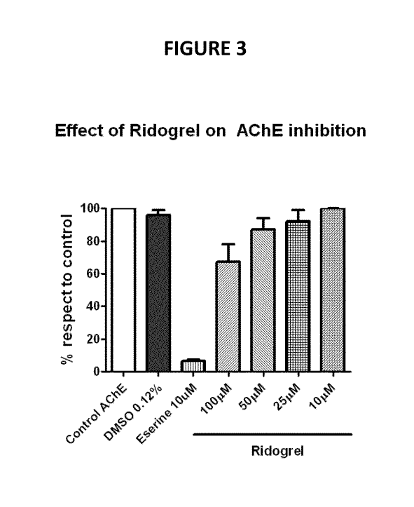

Ridogrel, a dual action drug used in prevention of systemic thrombo-embolism and an adjunctive agent to thrombolytic therapy in acute myocardial infarction, has shown to have a close relationship with neuronal dysfunction and cell death motive. Current therapy for AD is based on improving the brain synaptic availability of acetylcholine by using acetylcholinesterase inhibitors (AChEls) [17]. Therefore, potential drug effect on neuronal dysfunction was studied with an in vitro Acetylcholinesterase (AChE) assay using Amplex Red Acetylcholine/Assay Kit (Invitrogen, Carlsbad, Calif.) [18]. The efficacy of ridogrel on memory was obtained running dose-response (10-100 μM). 10 μM Eserine, an anticholinesterase drug, was used as positive control. Ridogrel has shown a dose-dependent inhibition of AChE with dose 100 to 25 μM, which highlights its efficacy on memory (FIG. 3).

### Example 8

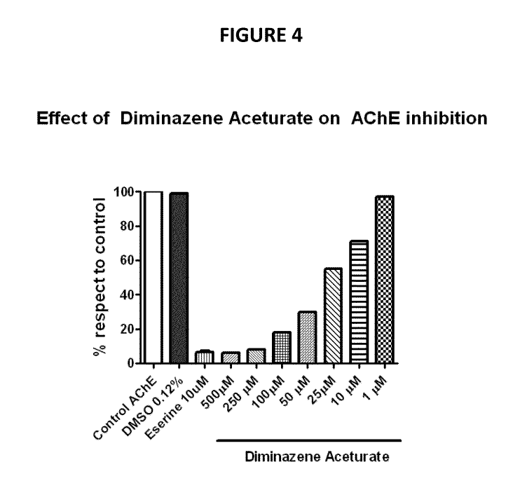

Diminazene aceturate, an effective trypanocidal agent has shown to have a close relationship with neuronal dysfunction and cell death motive. Current therapy for AD is based on improving the brain synaptic availability of acetylcholine by using acetylcholinesterase inhibitors (AChEls) [17]. Therefore, potential drug effect on neuronal dysfunction was studied with an in vitro Acetylcholinesterase (AChE) assay using Amplex Red Acetylcholine/Assay Kit (Invitrogen, Carlsbad, Calif.) [18]. The efficacy of diminazene aceturate on memory was obtained running dose-response (1-500 μM). 10 μM Eserine, an anticholinesterase drug, was used as positive control. Diminazene Aceturate has shown a dose-dependent inhibition of AChE with dose 500 to 10 μM, which highlights its efficacy on memory (FIG. 4).

### Example 9

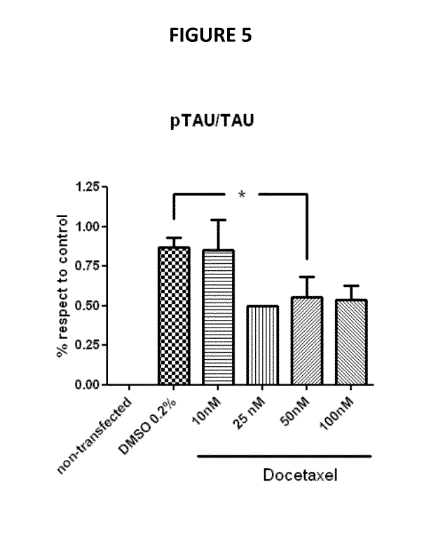

Docetaxel is a clinically well established anti-mitotic chemotherapy medication used mainly for the treatment of breast, ovarian and non-small cell lung cancer which had shown close proximity with Tau motive. Neurofibrillary tangles, a hallmark of AD, are intracellular abnormally aggregates of hyperphosphorylated protein TAU [19]. In that sense, Tau pathology was evaluated on tau-transfected in a mouse hippocampal-derived HT4 cell line using a phospho-tau and Tau ELISA assay (Sigma-aldrich, St Louis, Mo.). The level of TAU phosphorylation is used as an indicator of the degree of TAU pathology. Our results had shown that docetaxel treatment reduces TAU phosphorylation which is an indicator of the efficacy of the drug on TAU motive (FIG. 5).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### BIBLIOGRAPHY

1. MeSH, Medical Subject Headings. 2009.
2. Brookmeyer, R., et al., Forecasting the global burden of Alzheimer's disease. Alzheimers Dement, 2007. 3(3): p. 186-91.
3. Brodaty, H., et al., The World of Dementia Beyond 2020. J Am Geriatr Soc, 2011. 59(5): p. 923-927.
4. Lleo, A., Current therapeutic options for Alzheimer's disease. Curr Genomics, 2007. 8(8): p. 550-8.
5. Pujol, A., et al., Unveiling the role of network and systems biology in drug discovery. Trends Pharmacol Sci, 2010. 31(3): p. 115-23.
6. Aloy, P. and R. Russell, Targeting and tinkering with interaction networks. FEBS Lett, 2008. 582(8): p. 1219.
7. Jia, J., et al., Mechanisms of drug combinations: interaction and network perspectives. Nat Rev Drug Discov, 2009. 8(2): p. 111-28.
8. Nelson, H. S., Advair: combination treatment with fluticasone propionate/salmeterol in the treatment of asthma. J Allergy Clin Immunol, 2001. 107(2): p. 398-416.
9. Gupta, E. K. and M. K. Ito, Lovastatin and extended-release niacin combination product: the first drug combination for the management of hyperlipidemia. Heart Dis, 2002. 4(2): p. 124-37.
10. Larder, B. A., S. D. Kemp, and P. R. Harrigan, Potential mechanism for sustained antiretroviral efficacy of AZT-3TC combination therapy. Science, 1995. 269(5224): p. 696-9.
11. Dancey, J. E. and H. X. Chen, Strategies for optimizing combinations of molecularly targeted anticancer agents. Nat Rev Drug Discov, 2006. 5(8): p. 649-59.
12. Azmi, A. S., et al., Network perspectives on HDM2 inhibitor chemotherapy combinations. Curr Pharm Des, 2011. 17(6): p. 640-52.
13. Coma M, N. J., Systems biology upends drug reprofiling. Genetic Engineering and Biotechnology News, 2010. 30.
14. Pache R A, Z. A., Naval J, Mas J M, Aloy P, Towards a molecular characterisation of pathological pathways. FEBS Lett, 2008. 582: p. 6.
15. Selkoe, D. J., Biochemistry and molecular biology of amyloid beta-protein and the mechanism of Alzheimer's disease. Handb Clin Neurol, 2008. 89: p. 245-60.
16. Oddo, S., et al., Triple-transgenic model of Alzheimer's disease with plaques and tangles: intracellular Abeta and synaptic dysfunction. Neuron, 2003. 39(3): p. 409-21.
17. Doody, R. S., et al., Practice parameter: management of dementia (an evidence-based review). Report of the Quality Standards Subcommittee of the American Academy of Neurology. Neurology, 2001. 56(9): p. 1154-66.
18. Miao, Y., N. He, and J. J. Zhu, History and new developments of assays for cholinesterase activity and inhibition. Chem. Rev. 110(9): p. 5216-34.
19. Lee, V. M., Biomedicine. Tauists and beta-aptists united—well almost! Science, 2001. 293(5534): p. 1446-7.

What is claimed is:

1. A pharmaceutical composition comprising a therapeutically-effective amount of at least two or more compounds or an acceptable salt thereof, selected from the group including L-Glutamine, Biotin, L-Lysine, Vitamin C, L-Leucine, L-Methionine, L-Alanine, L-Isoleucine, Methadone, Methoxyflurane, Tacrolimus, Alfentanil, Aspirin, Halothane, Danazol, Estriol, Acetic Acid, Adenosine monophosphate, Arsenic trioxide, Atropine, Azelaic Acid, Chloroprocaine, Dimethyl sulfoxide, Ethanol, Fludarabine, Fomepizole, Isoflurane, L-Carnitine, Praziquantel, Promethazine, Rifampin, Spermine, Terfenadine, Vitamin E, Acarbose, Acetohydroxamic Acid, Aciclovir, Adenine, Adenosine triphosphate, Alclometasone, Alemtuzumab, Alendronate, Alpha-Linolenic Acid, Amifostine, Amlexanox, Amlodipine, Amodiaquine, Amrinone, Aspartame, Astemizole, Atazanavir, Atorvastatin, Atovaquone, Bacitracin, Balsalazide, Beclomethasone, Buclizine, Calcitriol, Cefadroxil, Cefalotin, Cefazolin, Cefdinir, Cefepime, Cefonicid, Cefoperazone, Cetuximab, Chloramphenicol, Chlorpheniramine, Cinnarizine, Ciprofloxacin, Clofarabine, Clopidogrel, Clotrimazole, Cloxacillin, Cocaine, Cyanocobalamin, Cyclizine, Cycloserine, Cyclosporine, Cyclothiazide, Cyproheptadine, Dapsone, Daunorubicin, Diethylstilbestrol, Dipyridamole, Disulfuram, Dofetilide, Enflurane, Enfuvirtide, Enoxacin, Enprofylline, Epinastine, Ertapenem, Ethchlorvynol, Ezetimibe, Felodipine, Fenofibrate, Flucytosine, Flumethasone Pivalate, Flunisolide, Fluorouracil, Fluticasone Propionate, Folic Acid, Framycetin, gamma-Homolinolenic acid, Gatifloxacin, Gemcitabine, Gentamicin, Glibenclamide, Glucosamine, Glutathione, Gonadorelin, Guanidine, Halobetasol Propionate, Halofantrine, Heparin, Hesperetin, Histamine Phosphate, Human Serum Albumin, Hyaluronidase, Hydroxocobalamin, Ibutilide, Icosapent, Iloprost, Imatinib, Irinotecan, Isoniazid, Isradipine, Itraconazole, L-Arginine, L-Aspartic Acid, L-Citrulline, L-Cysteine, L-Cystine, Levamisole, Levocabastine, Levofloxacin, Levothyroxine, Lidocaine, Lomefloxacin, L-Ornithine, L-Proline, L-Threonine, Menadione, Miconazole, Minocycline, Mitotane, Montelukast, Moxifloxacin, Mycophenolic acid, Nafarelin, Nedocromil, Niacin, Nitrendipine, Nitrofurazone, Nitroglycerin, Norfloxacin, Ofloxacin, Olopatadine, Oseltamivir, Palivizumab, Pefloxacin, Pegademase bovine, Penicillamine, Phentolamine, Potassium Chloride, Pranlukast, Pravastatin, Probenecid, Procaine, Proguanil, Pyrazinamide, Pyrimethamine, Pyruvic acid, Quinacrine, Quinine, Ribavirin, Riboflavin, Rifabutin, Rimexolone, Roxithromycin, Saquinavir, Sevoflurane, Sparfloxacin, Succinic acid, Sulfinpyrazone, Sulfisoxazole, Sulindac, Tetracycline, Tetrahydrofolic acid, Theophylline, Topotecan, Tretinoin, Triamcinolone, Trifluridine, Trimethoprim, Trovafloxacin, Urokinase, Verapamil, Vidarabine, Vitamin A, Vorinostat, Warfarin, Xanthophyll, Zanamivir, L-Histidine, Choline, Glycine, L-Tryptophan, L-Serine, NADH, Tramadol, Caffeine, Lisdexamfetamine, Methamphetamine, Methoxamine, Prazosin, Lorazepam, Butorphanol, Codeine, Hydrocodone, Hydromorphone, Morphine, Nalbuphine, Oxycodone, Propoxyphene, Digoxin, Progesterone, Meperidine, Sotalol, Dextromethorphan, Anileridine, Diphenoxylate, Levomethadyl Acetate, Levorphanol, Methadyl Acetate, Oxymorphone, Remifentanil, Sufentanil, Apomorphine, Malathion, Buprenorphine, Pentazocine, Magnesium Sulfate, Thiopental, Hydrocortisone, Iron Dextran, Estradiol, Perphenazine, Fentanyl, Propofol, Naloxone, Estramustine, Finasteride, Paclitaxel, Dexamethasone, Acamprosate, Allopurinol, Aminocaproic Acid, Amoxapine, Bambuterol, Colchicine, Dasatinib, Diazepam, Ethopropazine, Haloperidol, Ketamine, L-Glutamic Acid, Loperamide, L-Phenylalanine, Orphenadrine, Oxprenolol, Raloxifene, Rasagiline, Trandolapril, Tranexamic Acid, Acebutolol, Acenocoumarol, Acetaminophen, Acetazolamide, Acetophenazine, Adenosine, Adinazolam, Alfuzosin, Almitrine, Alosetron, Alprazolam, Alprenolol, Alteplase, Aluminium, Amantadine, Amiloride, Aminolevulinic acid, Amiodarone, Amitriptyline, Amphetamine, Anakinra, Anisotropine Methylbromide, Aprepitant, Aprindine, Argatroban, Aripiprazole, Atenolol, Atomoxetine, Azacitidine, Benazepril, Benzphetamine, Benzquinamide, Benzthiazide, Benztropine, Bepridil, Betaxolol, Bethanechol, Bethanidine, Bevantolol, Bezafibrate, Biperiden, Bisoprolol, Brimonidine, Brinzolamide, Bromocriptine, Budesonide, Bumetanide, Bupivacaine, Bupropion, Buspirone, Butabarbital, Butalbital, Butethal, Cabergoline, Candesartan, Capecitabine, Carbamazepine, Carbetocin, Carbidopa, Carbinoxamine, Carmustine, Carphenazine, Carvedilol, Ceftriaxone, Celecoxib, Chlordiazepoxide, Chlormerodrin, Chlormezanone, Chloroquine, Chlorpromazine, Chlorprothixene, Chlorthalidone, Chlorzoxazone, Cholecalciferol, Ciclopirox, Cimetidine, Cinolazepam, Citalopram, Cladribine, Clenbuterol, Clobazam, Clomifene, Clomipramine, Clonazepam, Clonidine, Clorazepate, Clotiazepam, Clozapine, Corticotropin, Creatine, Cyclobenzaprine, Cyclopentolate, Cycrimine, Cysteamine, Cytarabine, Dacarbazine, Dapiprazole, Darbepoetin alfa, Debrisoquin, Decitabine, Desflurane, Desipramine, Dexfenfluramine, Dexmedetomidine, Dexrazoxane, Dextroamphetamine, Diazoxide, Diethylpropion, Diflunisal, Digitoxin, Dihydroergotamine, Divalproex sodium, Dobutamine, Docetaxel, Donepezil, Doxepin, Doxorubicin, Doxylamine, Droperidol, Duloxetine, Echothiophate Iodide, Eletriptan, Entacapone, Ephedrine, Epoetin alfa, Eprosartan, Ergoloid mesylate, Ergotamine, Erlotinib, Escitalopram, Esomeprazole, Estazolam, Estrone, Eszopiclone, Etanercept, Ethinamate, Ethosuximide, Ethotoin, Etomidate, Etoricoxib, Exenatide, Famotidine, Felbamate, Fencamfamine, Flavoxate, Floxuridine, Fluconazole, Fludrocortisone, Flumazenil, Flunarizine, Flunitrazepam, Fluoxetine, Flupenthixol, Fluphenazine, Flurazepam, Flurbiprofen, Fluspirilene, Flutamide, Fluvastatin, Fluvoxamine, Fosfomycin, Fosphenyloin, Frovatriptan, Gabapentin, Galantamine, Gefitinib, Ginkgo biloba, Ginseng, Glimepiride, Goserelin, Granisetron, Guanabenz, Guanethidine, Halazepam, Hexachlorophene, Hexobarbital, Homatropine Methylbromide, Hydralazine, Hyoscyamine, Ibuprofen, Idarubicin, Ifosfamide, Imipramine, Indomethacin, Insulin Lyspro recombinant, Insulin recombinant, Insulin, porcine, Interferon alfa-2a, Irbesartan, Isocarboxazid, Isofluorophate, Isoproterenol, Isosorbide Dinitrate, Isotretinoin, Ketoprofen, Ketorolac, Labetalol, Lamotrigine, Lansoprazole, Lapatinib, Latanoprost, Lenalidomide, Letrozole, Leucovorin, Leuprolide, Levallorphan, Levetiracetam, Levobunolol, Levobupivacaine, Levodopa, Liothyronine, Lipoic Acid, Lisinopril, Lisuride, Lithium, Lofexidine, Losartan, Lovastatin, Loxapine, L-Tyrosine, Lucanthone, Lutropin alfa, L-Valine, Maprotiline, Maraviroc, Marinol, Mazindol, Mecamylamine, Mecasermin, Mefenamic acid, Mefloquine, Megestrol, Melatonin, Meloxicam, Memantine, Mephenyloin, Mepivacaine, Meprobamate, Mercaptopurine, Mesoridazine, Metformin, Metharbital, Methazolamide, Methohexital, Methotrexate, Methotrimeprazine, Methyldopa, Methylphenidate, Methylphenobarbital, Methylprednisolone, Methyprylon, Methysergide, Metixene, Metoclopramide, Metoprolol, Metyrosine, Mexiletine, Mianserin, Midazolam, Mifepristone, Miglitol, Miglustat, Milnacipran, Minaprine, Mirtazapine, Modafinil, Molindone, Moricizine, Mycophenolate mofetil, Nabilone, Nadolol, Naltrexone, Nandrolone, Naproxen, Naratriptan, Natalizumab, Nefazodone, Nesiritide, Nicardipine, Nicergoline, Nicotine, Nisoldipine, Nitrazepam, Nitric Oxide, Nortriptyline, Olanzapine, Olmesartan, Omeprazole, Ondansetron, Orlistat, Ouabain, Oxazepam, Oxcarbazepine, Oxybutynin, Paliperidone, Palonosetron, Paramethadione, Paroxetine, Pemetrexed, Pentobarbital, Pentostatin, Pergolide, Perindopril, Phenelzine, Phenmetrazine, Phenobarbital, Phenoxybenzamine, Phentermine, Phenylbutazone, Phenylpropanolamine, Phenyloin, Phosphatidylserine, Physostigmine, Picrotoxin, Pilocarpine, Pimozide, Pindolol, Pirbuterol, Piroxicam, Pramipexole, Prazepam, Prednisone, Pregabalin, Prilocalne, Primidone, Procainamide, Prochlorperazine, Procyclidine, Progabide, Promazine, Propericiazine, Propiomazine, Propranolol, Protriptyline, Pseudoephedrine, Pyridoxal, Quazepam, Quetiapine, Quinestrol, Quinidine, Ramelteon, Ramipril, Ranitidine, Ranolazine, Reboxetine, Remoxipride, Reserpine, Ridogrel, Riluzole, Rimonabant, Risperidone, Ritodrine, Rituximab, Rivastigmine, Rizatriptan, Ropinirole, Ropivacaine, S-Adenosylmethionine, Salbutamol, Salicyclic acid, Salmeterol, Salmon Calcitonin, Salsalate, Scopolamine, Secobarbital, Secretin, Selegiline, Sermorelin, Sertindole, Sertraline, Sibutramine, Simvastatin, Sirolimus, Sitagliptin, Sodium lauryl sulfate, Solifenacin, Somatropin recombinant, Sorafenib, Spirapril, Streptokinase, Sulfasalazine, Sulpiride, Sumatriptan, Sunitinib, Suramin, Tacrine, Talbutal, Tamibarotene, Tamoxifen, Telmisartan, Temazepam, Tetrabenazine, Tetrahydrobiopterin, Thalidomide, Thiabendazole, Thiamine, Thiethylperazine, Thioguanine, Thioridazine, Tiagabine, Tizanidine, Tocamide, Tolbutamide, Tolcapone, Tolmetin, Topiramate, Torasemide, Tranylcypromine, Trazodone, Triamterene, Triazolam, Trifluoperazine, Triflupromazine, Trihexyphenidyl, Trimethadione, Trimetrexate, Trimipramine, Tropicamide, Valproic Acid, Valrubicin, Valsartan, Vapreotide, Vasopressin, Venlafaxine, Vigabatrin, Vinblastine, Vindesine, Voriconazole, Yohimbine, Zaleplon, Ziprasidone, Zolmitriptan, Zolpidem, Zonisamide, Zopiclone, Zuclopenthixol, Diminazene Aceturate, Milnacipran, Amlodipine, Aranidipine, Azelnidipine, Barnidipine, Cilnidipine, Clevidipine, Efonidipine, Lacidipine, Lercanidipine, Manidipine, Nifedipine, Nilvadipine, Nimodipine, Nitrendipine, Nitrepin, Pranidipine, Diltiazem, Mibefradil, Clodronate, Etidronate, Tiludronate, Pamidronate, Neridronate, Olpadronate, Ibandronate, Risedronate, and Zoledronate.

2. A pharmaceutical composition comprising a therapeutically-effective amount of at least one calcium channel blocker and one bisphosphonate.

3. A pharmaceutical composition according to claim 2 where the calcium channel blocker is Amlodipine, Aranidipine, Azelnidipine, Barnidipine, Benidipine, Cilnidipine, Clevidipine, Isradipine, Efonidipine, Felodipine, Lacidipine, Lercanidipine, Manidipine, Nicardipine, Nifedipine, Nilvadipine, Nimodipine, Nisoldipine, Nitrendipine, Nitrepin, Pranidipine, Verapamil, Diltiazem, Mibefradil, Bepridil, Fluspirilene, and Fendiline or an acceptable salt thereof.

4. A pharmaceutical composition according to claim 2 where the bisphosphonate is Etidronate, Clodronate, Tiludronate, Pamidronate, Neridronate, Olpadronate, Alendronate, Ibandronate, Risedronate, and Zoledronate, or an acceptable salt thereof.

5. A pharmaceutical composition comprising a therapeutically-effective amount of at least two or more compounds or an acceptable salt thereof, selected from the group including riluzol, bepridil, diazoxide, thiamine, methylsergide, minaprine, alendronate, miconazole, melatonin, docetaxel, tamibarotene, ridogrel and diminazene aceturate.

6. A pharmaceutical composition comprising a therapeutically-effective amount of at least two or more compounds or an acceptable salt thereof, selected from the group including ridogrel, diminazene aceturate, riluzol, bepridil, docetaxel and alendronate.

7. A pharmaceutical composition comprising a therapeutically-effective amount of at least bepridil and alendronate, or an acceptable salt thereof.

8. A pharmaceutical composition according to claims 1 to 7, further comprising at least one pharmaceutically acceptable carrier.

9. A pharmaceutical composition according to claims 1 to 8, further comprising a therapeutically-effective amount of at least one other agent useful to treat neurological diseases.

10. A method of treating a neurological disease in a patient comprising administering to the patient in need of such treatment a therapeutically effective amount of a pharmaceutical composition according to claims 1 to 9.

11. A method according to claim 11, wherein the neurological disease is a neurodegenerative disease.

12. A method according to claim 10, wherein the disease is Parkinson Disease, Tauopathies, Alzheimer's Disease, Diffuse Neurofibrillary Tangles with Calcification, Supranuclear Palsy, Progressive, TDP-43 Proteinopathies, Amyotrophic Lateral Sclerosis, Frontotemporal Lobar Degeneration, Lewy Body Disease, AIDS Dementia Complex, Aphasia, Primary Progressive, Primary Progressive Nonfluent Aphasia, Dementia, Vascular, CADASIL, Dementia, Multi-Infarct, Diffuse Neurofibrillary Tangles with Calcification, Frontotemporal Lobar Degeneration, Frontotemporal Dementia, Primary Progressive Nonfluent Aphasia, Kluver-Bucy Syndrome, Pick's Disease, Motor Neuron Disease, Amyotrophic Lateral Sclerosis, Bulbar Palsy, Progressive, Muscular Atrophy, Spinal, Multiple System Atrophy, Olivopontocerebellar Atrophies, Shy-Drager Syndrome, Striatonigral Degeneration, Olivopontocerebellar Atrophies, Paraneoplastic Syndromes, Nervous System, Lambert-Eaton Myasthenic Syndrome, Limbic Encephalitis, Myelitis, Transverse, Opsoclonus-Myoclonus Syndrome, Paraneoplastic Cerebellar Degeneration, Paraneoplastic Polyneuropathy, Postpoliomyelitis Syndrome, Prion Diseases, Encephalopathy, Bovine Spongiform, Gerstmann-Straussler-Scheinker Disease, Insomnia, Fatal Familial, Kuru, Scrapie, Wasting Disease, Chronic, Creutzfeldt-Jakob Syndrome, Shy-Drager Syndrome, Subacute Combined Degeneration, Heredodegenerative Disorders, Nervous System, Alexander Disease, Amyloid Neuropathies, Familial, Bulbo-Spinal Atrophy, X-Linked, Canavan Disease, Cockayne Syndrome, Dystonia Musculorum Deformans, Gerstmann-Straussler-Scheinker Disease, Hepatolenticular Degeneration, Hereditary Central Nervous System Demyelinating Diseases, Hereditary Sensory and Autonomic Neuropathies, Hereditary Sensory and Motor Neuropathy, Huntington Disease, Lafora Disease, Lesch-Nyhan Syndrome, Menkes Kinky Hair Syndrome, Myotonia Congenita, Myotonic Dystrophy, Neurofibromatoses, Neuronal Ceroid-Lipofuscinoses, Optic Atrophies, Hereditary, Pantothenate Kinase-Associated Neurodegeneration, Rett Syndrome, Spinal Muscular Atrophies of Childhood, Spinocerebellar Degenerations, Tourette Syndrome, Tuberous Sclerosis, or Unverricht-Lundborg Syndrome.

13. A method according to claim 10 wherein the disease is Alzheimer's Disease or associated dementias.

14. A method according to claim 10 wherein the disease is defined by the presence of at least one of the followings pathophysiological processes: Amyloid pathology, Tau pathology, Oxidative Stress and Neuronal dysfunction and death.

15. A method of preventing the onset of, or protecting a patient from, a neurological disease comprising administering to the patient in need of such treatment a therapeutically effective amount of a pharmaceutical composition according to claims 1 to 9.

16. A method according to claim 15 wherein the neurological disease is Alzheimer's Disease and associated dementias.

17. A method of treating or preventing early forms of cognitive impairment, memory loss, or mild dementia comprising administering to the patient in need of such treatment a therapeutically effective amount of a pharmaceutical composition according to claims 1 to 9.

18. A method of memory protection or preservation or cognitive level protection or preservation comprising administering to the patient in need of such treatment a therapeutically effective amount of a pharmaceutical composition according to claims 1 to 9.

19. A method of treating a neurological disease according to claims 10 to 18, wherein the pharmaceutical compositions according to claims 1 to 9 are administered simultaneously, sequentially or separately to a subject in need thereof.

20. A kit comprising, in one or more containers, separately or in admixture, one or more pharmaceutical compositions according to claims 1 to 9.
